SEQUENCE LISTING (23 sequences)

SEQ ID NO: 1 - protein, 490 aa, Artificial Sequence
  Synthetic Construct
MGILTSFGDDMWLLTTLLLWVPVGGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHPGPSKPFWVLVVVGGVLACYSLLVTVAFIIFWVRSKRSRLLHSDYMNMTPRRPGPTRKHYQPYAPPRDFAAYRSVDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 25 aa, Artificial Sequence
  Synthetic Construct
MGILTSFGDDMWLLTTLLLWVPVGG

SEQ ID NO: 3 - protein, 277 aa, Artificial Sequence [sequence omitted]

SEQ ID NO: 4 - protein, 6 aa, Artificial Sequence
  Synthetic Construct
PGPSKP

SEQ ID NO: 5 - protein, 27 aa, Artificial Sequence
  Synthetic Construct
FWVLVVVGGVLACYSLLVTVAFIIFWV

SEQ ID NO: 6 - protein, 41 aa, Artificial Sequence
  Synthetic Construct
RSKRSRLLHSDYMNMTPRRPGPTRKHYQPYAPPRDFAAYRS

SEQ ID NO: 7 - protein, 112 aa, Artificial Sequence
  Synthetic Construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 480 aa, Artificial Sequence
  Synthetic Construct
MGFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHPGPSKPFWVLVVVGGVLACYSLLVTVAFIIFWVRSKRSRLLHSDYMNMTPRRPGPTRKHYQPYAPPRDFAAYRSVDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 15 aa, Artificial Sequence
  Synthetic Construct
MGFLTTLLLWVPVDG

SEQ ID NO: 10 - protein, 277 aa, Artificial Sequence [sequence omitted]

SEQ ID NO: 11 - protein, 6 aa, Artificial Sequence
  Synthetic Construct
PGPSKP

SEQ ID NO: 12 - protein, 27 aa, Artificial Sequence
  Synthetic Construct
FWVLVVVGGVLACYSLLVTVAFIIFWV

SEQ ID NO: 13 - protein, 41 aa, Artificial Sequence; identical to SEQ ID NO: 6
  Synthetic Construct

SEQ ID NO: 14 - protein, 112 aa, Artificial Sequence; identical to SEQ ID NO: 7
  Synthetic Construct

SEQ ID NO: 15 - protein, 469 aa, Artificial Sequence
  Synthetic Construct
MGFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIVRSKRSRLLHSDYMNMTPRRPGPTRKHYQPYAPPRDFAAYRSVDRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 15 aa, Artificial Sequence
  Synthetic Construct
MGFLTTLLLWVPVDG

SEQ ID NO: 17 - protein, 277 aa, Artificial Sequence [sequence omitted]

SEQ ID NO: 18 - protein, 21 aa, Artificial Sequence
  Synthetic Construct
VLFYLAVGIMFLVNTVLWVTI

SEQ ID NO: 19 - protein, 41 aa, Artificial Sequence; identical to SEQ ID NO: 6
  Synthetic Construct

SEQ ID NO: 20 - protein, 112 aa, Artificial Sequence
  Synthetic Construct
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 22 - protein, 22 aa, Artificial Sequence
  Synthetic Construct
VLFYLAVGIMFLVNTVLWVTIV

SEQ ID NO: 23 - protein, 155 aa, Artificial Sequence
  Synthetic Construct
RSKRSRLLHSDYMNMTPRRPGPTRKHYQPYAPPRDFAAYRSVDRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2024/0082400 A1
(43) Pub. Date: Mar. 14, 2024

(54) CD64 CHIMERIC RECEPTOR AND USES THEREOF

(71) Applicants: THE GENERAL HOSPITAL CORPORATION, Boston, MA (US); CONSIGLIO NAZIONALE DELLE RICERCHE, Rome (IT)

(72) Inventors: Soldano FERRONE, Boston, MA (US); Giuseppe SCONOCCHIA, Rome (IT); Sara CARATELLI, Rome (IT)

(21) Appl. No.: 18/272,168

(22) PCT Filed: Jan. 18, 2022
(86) PCT No.: PCT/US2022/012815

§ 371 Date: Jul. 13, 2023

Related U.S. Application Data
(60) Provisional application No. 63/138,152, filed on Jan. 15, 2021.

(51) Int. Cl. A61K 39/00; A61K 39/395; A61P 35/00; C07K 14/705; C07K 14/725; C07K 14/735; C12N 5/0783

(52) U.S. Cl. CPC A61K 39/4631; A61K 39/3955; A61K 39/4611; A61P 35/00; C07K 14/7051; C07K 14/70521; C07K 14/70535; C12N 5/0636; C07K 2317/24; C07K 2319/03; C07K 2319/30; C12N 2510/00

(57) ABSTRACT

The present invention provides compositions and methods for treating cancer in a human. The invention includes a chimeric receptor which comprises an CD64 binding domain, a transmembrane domain, and a CD3zeta signaling domain.

### CROSS-REFERENCE TO RELATED APPLICATIONS

This application claims benefit of U.S. Provisional Application No. 63/138,152, filed on Jan. 15, 2021, the contents of which are incorporated herein by reference in their entirety.

### BACKGROUND OF THE INVENTION

The invention relates to the field of chimeric receptors and immunotherapy.

The therapeutic effectiveness of monoclonal antibodies (mAbs) in the treatment of solid and hematologic malignancies represents a revolutionary breakthrough in the field of cancer immunotherapy. mAbs exert their anti-tumor activity directly by interfering with crucial signaling pathways involved in tumor sustainment and indirectly by redirecting cytotoxic cells toward cancer cells, leading to the generation of the antibody-dependent cellular cytotoxicity (ADCC). Although several classes of immunoglobulins are capable of mediating ADCC, IgG is the predominant subclass used in mAb immunotherapy. Most of the current therapeutic IgGs employed in treating malignancies act through an Fcγ receptor (FcγR)-dependent mechanism. The FcγR family includes activating FcγRI (CD64), FcγRIIA (CD32A), FcγRIIIA (CD16A), and inhibitory FcγRIIB (CD32B) members, differentially expressed on blood cells.

Identification of compositions and methods for enhancing ADCC are needed for cancer treatments. The present invention addresses this need.

### SUMMARY OF THE INVENTION

The invention features, inter alia, (i) chimeric receptors including an extracellular ligand binding domain of CD64 (CD64 CR); (ii) nucleic acids, vectors, and cells including such chimeric receptors; (iii) pharmaceutical compositions including such nucleic acids and vectors encoding CD64 CRs, or cells including CD64 CRs; and (iv) and methods for using such compositions, including methods for treating cancer.

Surprisingly, recombinant constructs containing a longer murine leader sequence (LS) instead of the shorter human LS were efficiently expressed in human T lymphocytes after retroviral transduction.

In one aspect, the invention features a chimeric receptor including a.) an extracellular ligand binding domain of CD64; b.) a transmembrane domain; and c.) an intracellular domain.

In another aspect, the invention features a chimeric receptor including a.) an extracellular ligand binding domain of CD64, wherein the extracellular ligand binding domain of CD64 includes an amino acid sequence having at least 80%, at least 85%, at least 90%, at least 95%, at least 96%, at least 97%, at least 98%, or at least 99% sequence identity to the amino acid sequence of SEQ ID NO:3; b.) a transmembrane domain; and c.) an intracellular domain.

In another aspect, the invention features a chimeric receptor including a.) an extracellular ligand binding domain of CD64, wherein the extracellular ligand binding domain of CD64 includes one or more of the following: (i) a CD64 D2 domain C-strand (Tyr-133-Leu-136); (ii) a CD64 C′-strand (Lys-142-His-148); and/or (iii) a CD64 C′E loop (His-148-Trp-149); b.) a transmembrane domain; and c.) an intracellular domain.

In some embodiments of any of the preceding aspects, the CD64 D2 domain C-strand includes amino acid residues Tyr-133 to Leu-136 of CD64 (YNVL).

In some embodiments of any of the preceding aspects, the CD64 C′-strand includes amino acid residues Lys-142 to His-148 of CD64 (KAFKFFH).

In some embodiments of any of the preceding aspects, the CD64 C′E loop includes amino acid residues His-148 and Trp-149 of CD64 (HW).

In some embodiments of any of the preceding aspects, the extracellular ligand binding domain of CD64 includes (i) and (ii), (ii) and (iii), (i) and (iii), or (i), (ii), and (iii).

In some embodiments of any of the preceding aspects, the numbering of the amino acid residues is relative to SEQ ID NO:21.

In some embodiments of any of the preceding aspects, the extracellular ligand binding domain of CD64 includes an amino acid sequence having at least 80%, at least 85%, at least 90%, at least 95%, at least 96%, at least 97%, at least 98%, or at least 99% sequence identity to the amino acid sequence of SEQ ID NO:3.

In some embodiments of any of the preceding aspects, the chimeric receptor further includes a signal peptide.

In some embodiments of any of the preceding aspects, the signal peptide is a mouse CD64 signal peptide.

In some embodiments of any of the preceding aspects, the mouse CD64 signal peptide is humanized.

In some embodiments of any of the preceding aspects, the extracellular ligand binding domain includes D1, D2, and D3 of CD64.

In some embodiments of any of the preceding aspects, the transmembrane domain includes a CD64 transmembrane domain.

In some embodiments of any of the preceding aspects, the transmembrane domain further includes a hinge region.

In some embodiments of any of the preceding aspects, the transmembrane domain includes a CD28 transmembrane domain.

In some embodiments of any of the preceding aspects, the intracellular domain includes a CD28 intracellular domain.

In some embodiments of any of the preceding aspects, the intracellular domain includes a CD28-CD3 intracellular domain.

In some embodiments of any of the preceding aspects, the chimeric receptor includes CDK In some embodiments of any of the preceding aspects, the chimeric receptor binds to a molecule including Fc.

In some embodiments of any of the preceding aspects, the molecule including Fc is an IgG antibody.

In some embodiments of any of the preceding aspects, the IgG antibody is IgG1 or IgG3.

In another aspect, the invention features a nucleic acid including a nucleotide sequence encoding any one of the chimeric receptors disclosed herein.

In some embodiments of any of the preceding aspects, the nucleic acid is an RNA or a cDNA molecule.

In another aspect, the invention features a vector including any one of the nucleic acids disclosed herein.

In some embodiments of any of the preceding aspects, the vector is an expression vector.

In some embodiments of any of the preceding aspects, the vector is a viral vector.

In some embodiments of any of the preceding aspects, the viral vector is a retroviral vector.

In another aspect, the invention features a cell, including any one of the nucleic acids disclosed herein or any one of the vectors disclosed herein.

In some embodiments of any of the preceding aspects, the cell is an immune cell.

In some embodiments of any of the preceding aspects, the immune cell is a T cell, a natural killer cell, a macrophage, a neutrophil, an eosinophil, or a combination thereof.

In some embodiments of any of the preceding aspects, the cell is a T cell.

In another aspect, the invention features a pharmaceutical composition, including (a) any one of the nucleic acids disclosed herein, any one of the vectors disclosed herein, or any one of the cells disclosed herein, and (b) a pharmaceutically acceptable carrier.

In some embodiments of any of the preceding aspects, the composition further includes an Fc-containing therapeutic agent.

In some embodiments of any of the preceding aspects, the Fc-containing therapeutic agent is an Fc fusion protein or an antibody.

In some embodiments of any of the preceding aspects, the antibody binds to a cell surface antigen.

In some embodiments of any of the preceding aspects, the antibody binds epidermal growth factor receptor.

In some embodiments of any of the preceding aspects, the antibody is Cetuximab.

In some embodiments of any of the preceding aspects, the antibody binds B7-H3.

In some embodiments of any of the preceding aspects, the antibody is anti-B7-H3 376.96 mAb.

In some embodiments of any of the preceding aspects, the antibody is Enoblituzumab.

In another aspect, the invention features a kit, including: a first pharmaceutical composition that includes (i) any one of the nucleic acids disclosed herein, any one of the vectors disclosed herein, or any one of the cells disclosed herein, and (ii) a pharmaceutically acceptable carrier.

In some embodiments of any of the preceding aspects, the kit further includes a second pharmaceutical composition that includes an Fc-containing therapeutic agent and a pharmaceutically acceptable carrier.

In some embodiments of any of the preceding aspects, the Fc-containing therapeutic agent is an Fc fusion protein or an antibody.

In some embodiments of any of the preceding aspects, the antibody binds to a cell surface antigen.

In some embodiments of any of the preceding aspects, the antibody binds epidermal growth factor receptor.

In some embodiments of any of the preceding aspects, the antibody is Cetuxirnab.

In some embodiments of any of the preceding aspects, the antibody binds B7-H3.

In some embodiments of any of the preceding aspects, the antibody is anti-B7-H3 376.96 mAb.

In some embodiments of any of the preceding aspects, the antibody is Enoblituzumab.

In another aspect, the invention features a pharmaceutical composition for use in killing tumor cells or for treating cancer in a subject, the pharmaceutical composition including an effective amount of immune cells that express any one of the chimeric receptors disclosed herein and a pharmaceutically acceptable carrier.

In another aspect, the invention features a pharmaceutical composition for use in enhancing antibody-dependent cell-mediated cytotoxicity (ADCC) or antibody-dependent cellular phagocytosis (ADCP) or for enhancing efficacy of an antibody-based immunotherapy in a subject, the pharmaceutical composition including an effective amount of immune cells that express any one of the chimeric receptors disclosed herein and a pharmaceutically acceptable carrier.

In some embodiments of any of the preceding aspects, the immune cells are natural killer cells, macrophages, neutrophils, eosinophils, T cells, or a combination thereof.

In some embodiments of any of the preceding aspects, the immune cell is a T cell.

In some embodiments of any of the preceding aspects, the host immune cells are autologous.

In some embodiments of any of the preceding aspects, the host immune cells are allogeneic.

In some embodiments of any of the preceding aspects, the immune cells are activated, expanded, or both ex vivo.

In some embodiments of any of the preceding aspects, the subject has been treated or is being treating with an Fc-containing therapeutic agent.

In some embodiments of any of the preceding aspects, the Fc-containing therapeutic agent is a therapeutic antibody or a Fc fusion protein.

In some embodiments of any of the preceding aspects, the Fc-containing therapeutic agent is an antibody.

In some embodiments of any of the preceding aspects, the antibody binds to a cell surface antigen.

In some embodiments of any of the preceding aspects, the antibody binds epidermal growth factor receptor.

In some embodiments of any of the preceding aspects, the antibody is Cetuximab.

In some embodiments of any of the preceding aspects, the antibody binds B7-H3.

In some embodiments of any of the preceding aspects, the antibody is anti-B7-H3 376.96 mAb.

In some embodiments of any of the preceding aspects, the antibody is Enoblituzumab.

In some embodiments of any of the preceding aspects, the subject is a human patient suffering from a cancer and the Fc-containing therapeutic agent is for treating the cancer.

In some embodiments of any of the preceding aspects, the cancer includes cells expressing epidermal growth factor.

In some embodiments of any of the preceding aspects, the cancer includes cells expressing B7-H3.

In some embodiments of any of the preceding aspects, the cancer is a solid cancer or a hematopoietic cancer.

In some embodiments of any of the preceding aspects, the cancer is a carcinoma, lymphoma, a sarcoma, blastoma, a leukemia, or a cancer stem cell.

In some embodiments of any of the preceding aspects, the cancer is acute myeloid leukemia, breast cancer, lung cancer, non small cell lung carcinoma, gastrointestinal cancer, colorectal cancer, head and neck cancer, and glioblastoma multiforme, and glioblastoma multiforme cancer stem cells.

In some embodiments of any of the preceding aspects, the subject's cancer has been irradiated or is being irradiated.

In some embodiments, the subject has one or more tumors and the immune cells migrate to the one or more tumors.

In another aspect, the invention features a method for preparing immune cells expressing a chimeric receptor, including: (i) providing a population of immune cells; (ii) introducing into the immune cells a nucleic acid encoding any one of the chimeric receptors disclosed herein; and (iii) culturing the immune cells under conditions allowing for expression of the chimeric receptor.

In some embodiments of any of the preceding aspects, the population of immune cells are derived from peripheral blood mononuclear cells (PBMC).

In some embodiments of any of the preceding aspects, the immune cells are natural killer cells, macrophages, neutrophils, eosinophils, T cells, or a combination thereof.

In some embodiments of any of the preceding aspects, the immune cells are derived from a human patient.

In some embodiments of any of the preceding aspects, the human patient is a cancer patient.

In some embodiments of any of the preceding aspects, the nucleic acid is a viral vector.

In some embodiments, the viral vector is a retroviral vector.

In some embodiments of any of the preceding aspects, the nucleic acid is an RNA molecule.

In some embodiments of any of the preceding aspects, the vector is introduced into the immune cells by retroviral transduction or electroporation.

In another aspect, the invention features a method of treating cancer in a subject, including administering to the subject an effective amount of any one of the chimeric receptors disclosed herein, any one of the nucleic acids disclosed herein, any one of the vectors disclosed herein, any one of the cells disclosed herein, and/or any one of the pharmaceutical compositions described herein, thereby treating cancer in the subject.

In another aspect, the invention features a method of enhancing ADCC or ADCP or for enhancing efficacy of an antibody-based immunotherapy in a subject having a cancer, including administering to the subject an effective amount of any one of the chimeric receptors disclosed herein, any one of the nucleic acids disclosed herein, any one of the vectors disclosed herein, any one of the cells disclosed herein, and/or any one of the pharmaceutical compositions described herein, thereby enhancing ADCC or ADCP or enhancing efficacy of the antibody-based immunotherapy in the subject.

In some embodiments of any of the preceding aspects, the subject has been treated or is being treating with an Fc-containing therapeutic agent.

In some embodiments of any of the preceding aspects, the Fc-containing therapeutic agent is a therapeutic antibody or a Fc fusion protein.

In some embodiments of any of the preceding aspects, the Fc-containing therapeutic agent is an antibody.

In some embodiments of any of the preceding aspects, the antibody binds to a cell surface antigen.

In some embodiments of any of the preceding aspects, the antibody binds epidermal growth factor receptor.

In some embodiments of any of the preceding aspects, the antibody is Cetuximab.

In some embodiments of any of the preceding aspects, the antibody binds B7-H3.

In some embodiments of any of the preceding aspects, the antibody is anti-B7-H3 376.96 mAb.

In some embodiments of any of the preceding aspects, the antibody is Enoblituzumab.

In some embodiments of any of the preceding aspects, the Fc-containing therapeutic agent is for treating the cancer.

In some embodiments of any of the preceding aspects, the cancer includes cells expressing epidermal growth factor.

In some embodiments of any of the preceding aspects, the cancer includes cells expressing B7-H3.

In some embodiments of any of the preceding aspects, the cancer is a solid cancer or a hematopoietic cancer.

In some embodiments of any of the preceding aspects, the cancer is a carcinoma, lymphoma, a sarcoma, blastoma, a leukemia, or a cancer stem cell.

In some embodiments of any of the preceding aspects, the subject's cancer has been or is being irradiated.

In some embodiments of any of the preceding aspects, the subject has one or more tumors and the immune cells migrate to the one or more tumors.

Other features and advantages of the invention will be apparent from the following description of the preferred embodiments thereof, and from the claims.

### BRIEF DESCRIPTION OF THE DRAWINGS

FIG. 1. Phenotypic features of the CD64 chimeric receptor (CR)-transduced T cells. A.) Schematic representation of four CD64-CR variants. All CD64 chimeric constructs shared the extracellular region of the human CD64. The first, (Ms-SP)-CD64.284, and the second, CD64.284, construct included CD28 transmembrane and cytoplasmic domain fused to the CD3 intracellular motif. They differed in the signal peptide (SP): in the first one the SP derived from mouse CD64 (Ms-SP) while in the second one the SP belonged to human CD64 (Hu-SP). In the third variant, the CD64(EC-TM).284, the human SP, the extracellular and the transmembrane regions of the CD64 were directly linked to the CD28 and CD3 intracellular signaling domains. EC: extracellular, TM: transmembrane, IC: intracellular. B.) Representative expression of the CD64-CR variants in human T cells. Activated human T cell blasts from the same donor were transduced with the four CD64-CR constructs. Fourteen days post transduction, T cells were collected and stained with FITC-conjugated anti-human CD3 and PE-conjugated anti-human CD64 mAbs and then analyzed by flow cytometry. Numbers in the quadrants indicate the percentage of cells. C.) Monitoring of the (Ms-SP)-CD64-CR (hereafter referred as CD64-CR) expression in T cells and its distribution on the CD4⁺ and CD8⁺ T cell subsets over time. CD64-CR T cells were stained at day 4, 11 and 18 post-transduction using FITC-conjugated anti-human CD3, PE-conjugated anti-human CD64, PerCP-Cy5.5-conjugated anti-human CD4 and APC-conjugated anti-human CD8 mAbs. Stained T cells were enumerated by flow cytometry and the percentages of cells are shown in the quadrants. D.) Cumulative analysis of the CD64-CR expression in T cells over time (N=5). T cells from five different donors (D1-5) were transduced with CD64-CR and the expression of the CR was analyzed by flow cytometry at 4-, 11-, 18- and 25-days post-transduction. At the indicated times, T cells were collected and stained with FITC-conjugated anti-human CD3 and PE-conjugated anti-human CD64. The percentage of CD64⁺CD3⁺ T cells is shown. E.) Representative four-color flow cytometry analysis of CD64-CR T cells at 30 days post transduction showing the frequency of stem cell memory T cells (CD45RA+CCR7+CD62L+), central memory T cells (CD45RA−CCR7+CD62L+), effector memory T cells (CD45RA−CCR7−CD62L−), and effector T cells (CD45RA+CCR7−CD62L−) in the CD64+CD3+ T cells.

FIG. 2. CD64-CR T cells directly eliminated KRAS-mutated CRC HCT-116 in vitro without requiring the presence of mAbs.

A.) Representative bioluminescence image (left panel) of firefly luciferase expressing HCT-116 Luc+ co-cultured with CD64-CR or non-transduced T effector cells at different E:T ratios. Each experimental condition was represented in triplicates. After a 72-hour co-culture, D-luciferin (150 μg/mL) was added to cell culture medium. Photons (total flux) emitted from HCT-116 Luc+ in the selected regions of interest (ROI) were quantified using the Living Image® software (right panel). ****p<0.0001, two-way ANOVA with Bonferroni test adjusted p value was used for statistical analysis. B.) Representative MTT assay showing viable HCT-116 and HT-29 tumor cells after a 72-hour co-culture with CD64-CR T or non-transduced (NT) T cells at different E:T ratios. Each condition was performed in triplicate and means±SD of optical density measured at 570 nM (Ab 570 nM) are shown. C.) Flow-cytometry plots of HCT-116 and HT-29 CRC cell lines after a 4-hour incubation with CD64-CR T cells at an E:T ratio of 2:1. Non-transduced (NT) T cells were used as a control. Following co-culture, the cells were collected and stained with APC-conjugated anti-human CD3 mAb, FITC-conjugated annexin V and PI. HCT-116 and HT-29 target cells were distinguished from T effector cell by posting an electronic gate on CD3− cells. Percentages of cells were indicated in the quadrants. D.) HCT-116 and HT-29 cell viability tested with trypan-blue exclusion cell count. CRC target cells were co-cultured with CD64-CR T or non-transduced (NT) T cells at the indicated E:T ratio. After an overnight-incubation at 37° C., non-adherent effector T cells were removed and the remaining adherent HCT-116 or HT-29 were counted. Error bars denote SD. Unpaired two-tailed T test was used; *p<0.05, **p<0.01.

FIG. 3. CD64-CR T cells efficiently killed CRC target cell lines and minimally affected non-tumor cell viability in vitro.

A.) Determination of residual BJ and HCT-116 cell number following an overnight-incubation with CD64-CR T cells at E:T ratios of 2:1 and 1:1. Non transduced (NT) T cells were used as a control. Error bars indicate SD. Unpaired two-tailed T test was applied; *p<0.05, **p<0.01. B.) Representative contour plots showing viability of human BJ normal fibroblasts (upper panel) or CRC HCT-116 cell line (lower panel) after a 4-hour co-culture of with or without CD64-CR T cells at an E:T ratio of 2:1. The CD3− target cell viability was assessed by annexin V and PI staining and following flow cytometry analysis. Numbers in the quadrants indicate percentage of cells. C.) Non-tumor BJ, IMR-90 fibroblasts, human myoblasts and CRC HCT-116 cells were co-cultured with CD64-CR T and non-transduced (NT) T cells at indicated E:T ratio for 72 hours. The viability of target cells was determined by MTT assay. Each point in the curves indicate mean±SD of absorbance detected at 570 nM (Ab₅₇₀) in four independent experiments (N=4) for BJ fibroblasts and HCT116 and two independent experiments (N=2) for IMR-90 and myoblasts. *p<0.05; **p<0.01; ****p<0.0001; two-way-ANOVA with Bonferroni test was used.

FIG. 4. The direct anti-CRC activity of CD64-CR T cells is enhanced by ADCC mechanism in in vitro co-cultures.

A.) HCT-116, HT-29 and CaCo-2 CRC cell lines were incubated with CD64-CR T or non-transduced (NT) T cells with or without cetuximab, panitumumab or 396.96 mAb (1 μg/ml) at different E:T ratio. Following a 72-hour co-culture, the tumor cell viability was assessed by MTT test. Three experimental replicates for each point was performed. Means and SD of the best of three independent donors (N=3) were showed. Ab₅₇₀: absorbance at 570 nM. B.) HCT-116 cells were co-cultured with or without CD64-CR T or non-transduced (NT) T cells at an E:T ratio of 2:1 for 4 hours at 37° C. in the presence or absence of 1 μg/ml of anti-EGFR mAb cetuximab or anti-B7-H3 mAb 376.96. Then, cells were collected and stained with APC-conjugated anti-human CD3, FITC-conjugated annexin V and PI and analyzed by flow cytometry. The numbers in the quadrants indicate percentage of cells.

FIG. 5. CD107A and perforin release analysis in CD64-CR T cells cocultured with HCT116 cells.

A.) CD64-CR T cells were incubated for 4 hours with or without HCT116 CRC cells at an E:T ratio of 2:1 in the presence or absence of 1 μg/ml of cetuximab (C), panitumumab (P) or 376.96 mAb. FITC-conjugated mouse anti-human CD107A mAb and 2 μM of monensin were included during coculture. Then, cells were stained with PE-conjugated mouse anti-human CD64 and analyzed by flow cytometry. MFI=mean fluorescence intensity. Numbers in the quadrants indicate percentages of cells. B.) Colocalization and polarization of perforin on CD64-CR T cells upon conjugation with EGFR positive HCT-116 CRC cells. The indirect immunofluorescence method utilized for these type of experiments is detailed in the methods section. HCT-116 cells were incubated in the presence of CD64-CR T cells at an T:E ratio of 1:2, for 30 minutes at 37° C., formalin fixed, permeabilized, and incubated, overnight at 4° C., in the presence of a rabbit anti-human EGFR mAb or a mouse anti-human CD64 ab. The cell mix was incubated with a Cy-5-conjugated donkey anti-rabbit or a Cy3-conjugated donkey anti-mouse. Then, the cell mix was incubated, for 2 hours at 37° C., with a FITC-conjugated mouse anti-human perforin mAb. The cell mix nuclei were stained by DAPI. The Cells were analyzed as indicated by confocal microscopy.

FIG. 6. The CD64-CR was compared to the CD16^{158V}-CR and to the classical B7-H3.CAR.

A.) CD64-CR T cells exerted a superior anti-tumor activity than CD16^{158V}-CR T cells against HCT-116 cells. HCT-116 CRC cells were cocultured, in triplicate, with CD64-CR or CD16^{158V}-CR transduced T cells in the presence or absence of 1 μg/ml of cetuximab or 376.96 mAb at increasing E:T ratios. HCT-116 cell survival was analyzed by MTT assay after 72-hour incubation. Non transduced (NT) T cells were used as a control. Mean±SD are shown. *p<0.05, ****p<0.0001, two-way-ANOVA with Bonferroni test was applied. Ab₅₇₀: absorbance at 570 nM. ns: not significant. B.) B7-H3.CAR expression on human T lymphocytes. Activated CD3⁺ T cells were transduced with retroviral particle carrying the B7-H3.CAR vector. After a 3-day incubation, the expression of B7-H3.CAR was determined by flow cytometry; dotted line light gray histogram shows control non-transduced (NT) T cells, full line gray histogram indicates B7-H3.CAR transduced T cells. C.) Equivalent activity of CD64-CR T cells in combination with anti-B7-H3 mAb 376.96 and the classic B7-H3.CAR T cells against CRC cell lines. Triplicates of CD64-CR T cells with or without 1 μg/ml of 376.96 mAb or B7-H3.CAR T cells, obtained from the same donor and cultured in the same conditions, were incubated with HCT-116 or HT-29 target cells at different E:T ratios. Non-transduced (NT) T cells were used as a control. Following a 72-hour incubation at 37° C., MTT assay was performed to determine the viability of the HCT-116 and HT-29 remaining cells. The quantification of absorbance at 570 nM (Ab₅₇₀) was showed as mean±SD. ****p<0.0001, two-way-ANOVA with Bonferroni test was used.

FIG. 7. Analysis of CD64-CR binding capacity of soluble mAb with or without human plasma and characterization of the CD64-CR T cell anti-tumor activity in the presence of a third-party FcγR+CD14+ cells.

A.) Representative flow-cytometry plots showing cetuximab, panitumumab and 376.96 mAb binding on CD64-CR T cells. CD64-CR T cells were incubated with 10 μg/ml of cetuximab, panitumumab or 376.96 mAb for 30 min at 4° C. in the presence or absence of FcR blocking reagent (BR). The binding of the indicated mAbs on T cell surface was revealed by staining cells with FITC-conjugated mouse anti-human IgG (MAH) (for cetuximab and panitumumab) or FITC-conjugated goat anti-mouse IgG (GAM) (for 376.96) for 30 min at 4° C. Numbers in the quadrants indicate percentage of cells. B.) Dose-response of the cetuximab, panitumumab and 376.96 mAb binding on CD64-CR T cells. CD64-CR transduced T cells were incubated with scaling amounts of cetuximab, panitumumab or 376.96 mAb. Following a 30 min-incubation at 4° C., cells were washed and stained with FITC-conjugated mouse anti-human IgG (MAH) or FITC-conjugated goat anti-mouse IgG (GAM) and analyzed by flow cytometry. The percentage of positive cells (left panel) and mean fluorescence intensity (MFI) (right panel) are shown. C.) Binding of the 376.96 mAb to CD64-CR T cells in the presence or absence of increasing concentrations of heat-inactivated human plasma at 37° C. and 4° C. CD64-CR T cells were incubated with 10 μg/ml of the 376.96 mAb at the mentioned temperatures. After a 30 min-incubation, T cells were stained with FITC-conjugated anti-mouse IgG at the same temperatures. The percentage of positive cells and the MFI were determined by flow cytometry. D.) The anti-CRC efficacy of the CD64-CR T cells was not affected by the presence of a third-party CD14+ monocytes. Seven days post transduction, CD64-CR T cells were incubated, in triplicate at 37° C., with HCT-116 cells and the indicated mAbs, in the presence or absence of a third party FcγR+CD14+ monocytes (M) at an effector:target:monocyte (E:T:M) ratio of 1:1:1. Following a 72-hour co-culture, non-adherent T cells were removed and the target cell viability was determined by MTT assay. The absorbance measured at 570 nm (Ab₅₇₀) is shown as mean±SD. *p<0.01, ****p<0.0001 were analyzed using two-way-ANOVA with Bonferroni test. NT: non transduced. See, also FIG. 4.

FIG. 8. Direct CD64-T cell cytotoxicity is antibody-independent.

FIG. 8 is a series of graphs showing a forty-eight-hour MTT cell viability assay of HCT116 cells incubated with CD64-CR T cells in 10% FCS, supplemented with scalar concentrations of human IgGs. Cancer cell lines, as indicated in the X-axes, were incubated at 37° C., with or without CD64-T cells, at an effector to target (E:T) ratio of 2:1.

FIG. 9. Effect of human serum on the anti-tumor activity of CD64-T cells.

FIG. 9 is a series of graphs showing the effect of human serum on the anti-tumor activity of CD64-T cells on HCT116 cells in the presence of the indicated concentrations of human serum, using 10% fetal bovine serum (FBS) medium as a positive control. CD64-T cells or non transduced T (NT T) cells were incubated at 37° C. with HCT116 cells at an E:T ratio of 2:1 with or without 1 μg/ml of cetuximab or 376.96 mAb, in the presence of 10% of FBS or 10%, 20% and 40% of heat-inactivated human serum (HS). After a 96-hour incubation, effector cells were removed and the viability of the remaining target cells was assessed by the MTT assay.

FIG. 10. Effect of irradiation on the anti-tumor activity of CD64-T cells.

FIG. 10 is a series of graphs showing the results of experiments in which A253 cells irradiated with 15 Gy, 10 Gy, or 5 Gy or non-irradiated were plated at a density of 20,000 cells/well in a 96 well flat bottom plate. CD64-CR T cells were added to target cells at 4:1 E:T ratio in the presence of RPMI medium supplemented with 10% of FBS, 10% or 40% of HS with or without 1 μg/ml of 376.96 mAb. After incubation, effector cells were removed and target cell viability was assessed by MTT assay.

FIG. 11. Effect of pH of the medium on the anti-tumor activity of CD64-T cells.

FIG. 11 is a series of graphs showing results obtained utilizing the MTT assay. CD64-T cells or NT T cells were incubated for 72 h with or without HCT116 cells, at the indicated E:T ratios. ADCC was assessed in the presence of the 376.96 mAb. The pH of the media varied as follows: A.) FBS 20%, pH 7.4, B.) FBS 20%, pH 6.8 (B); and C.) HS 20%, pH 6.8.

FIG. 12. Biodistribution of CD64-CR T cells in vivo.

A.) shows a schematic diagram showing the experiment described in Example 10. “Low fluo” indicates low fluorescence diet. B.) shows a series of images showing bioluminescent imaging (BLI) and fluorescence imaging (FLI) overlays for the indicated treatment groups for the experiment described in Example 10. These data show that both non-transduced (NT) T cells and CD64-CR T cells labeled with near-infrared cell tracker (NIR) migrate to the tumor site. C.) is a graph showing the BLI signal of cancer cells on day 2 of the experiment described in Example 10.

### DETAILED DESCRIPTION OF THE INVENTION

### Definitions

Unless defined otherwise, all technical and scientific terms used herein have the same meaning as commonly understood by one of ordinary skill in the art to which the invention pertains.

The articles “a” and “an” are used herein to refer to one or to more than one (i.e., to at least one) of the grammatical object of the article. By way of example, “an element” means one element or more than one element.

“Activation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, the term “Chimeric Receptor” or alternatively a “CR” refers to a recombinant polypeptide construct including at least an extracellular binding domain, a transmembrane domain and a cytoplasmic signaling domain including a functional signaling domain derived from a stimulatory molecule as defined below. In one aspect, the stimulatory molecule is the zeta chain associated with the T cell receptor complex.

As used herein, a “signaling domain” is the functional portion of a protein which acts by transmitting information within the cell to regulate cellular activity via defined signaling pathways by generating second messengers or functioning as effectors by responding to such messengers.

The term “anti-tumor effect” as used herein, refers to a biological effect which can be manifested by various means, including but not limited to, a decrease in tumor volume, a decrease in the number of tumor cells, a decrease in the number of metastases, an increase in life expectancy, a decrease in tumor cell proliferation, a decrease in tumor cell survival, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

As used herein, the term “autologous” is meant to refer to any material derived from the same individual to whom it is later to be re-introduced into the individual.

“Allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like, including other cancers described herein.

As used herein, the term “conservative sequence modifications” is intended to refer to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CD64 chimeric receptor can be replaced with other amino acid residues from the same side chain family and the altered receptor can be tested for the ability to bind, for example, Fc, using the standard functional assays.

As used herein, the term “percent (%) sequence identity,” with respect to a reference polynucleotide or polypeptide sequence, is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

A “stimulatory molecule,” means a molecule expressed by a T cell that provide the primary cytoplasmic signaling sequence(s) that regulate primary activation of the TCR complex in a stimulatory way for at least some aspect of the T cell signaling pathway. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences that are of particular use in the invention include those derived from TCR zeta, FcR gamma, FcR beta, CD3 gamma, CD3 delta, CD3 epsilon, CD5, CD22, CD79a, CD79b, CD278 (also known as “ICOS”) and CD66d. In a specific CR of the invention, the cytoplasmic signaling molecule in any one or more CRs of the invention, including CRs includes a cytoplasmic signaling sequence derived from CD3-zeta.

A “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class I molecule, BTLA and a Toll ligand receptor, as well as OX40, CD27, CD28, CDS, ICAM-1, LFA-1 (CD1 Ia/CD 18) and 4-1BB (CD137).

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i. e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can include a protein's or peptide's sequence. Polypeptides include any peptide or protein including two or more amino acids joined to each other by peptide bonds.

As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody or antibody fragment which recognizes and binds with a specific antigen, but does not substantially recognize or bind other molecules in a sample.

The term “therapeutic” as used herein means a treatment and/or prophylaxis. A therapeutic effect is obtained by suppression, remission, or eradication of a disease state.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into a host cell. A “transfected” or “transformed” or “transduced” cell is one which has been transfected, transformed or transduced with exogenous nucleic acid. The cell includes the primary subject cell and its progeny.

A “vector” is a composition of matter which includes an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

As is discussed below, we have designed a CD64-chimeric receptor (CD64-CR) composed of the human FcγRI extracellular binding domain linked to an intracellular tail capable of inducing a cytotoxic T cell response upon stimulation. The CD64-recombinant receptor was expressed on human T cells and its anti-tumor activity was tested, in vitro and in vivo, against colorectal carcinoma (CRC) cell lines in the presence or absence of anti-epidermal growth factor receptor (EGFR) mAbs or anti-B7-H3 mAb (376.96). The CD64-CR engineered T cells were efficiently redirected against CRC tumor cells and an MHC-unrestricted target cell killing was observed regardless of antibody ligation.

Fcγ-chimeric receptors (CRs), in particular, have been designed to redirect T cells against cancer cells by monoclonal antibodies (mAbs) with specificity for cell surface tumor-associated-antigens. Since FcγRs bind the Fc fragment of immunoglobulins G (IgGs), the antibody-dependent-cellular-cytotoxicicty (ADCC) is the expected mechanism by which Fcγ-CR T cells kill cancer cells. However, recent evidence suggests that, other then the Fc fragment of IgG, FcγRs bind also pentraxins. The aim of this study is to investigate whether Fcγ− (CD64) T cells act as biosensors capable of detecting unknown FcγR ligand(s) on the cell surface of cancer cells. Healthy, peripheral blood (PB) T cells were transduced with a retroviral vector coding for a human CD64-CR in which the human leader sequence (LS) was replaced with a mouse LS (mLS-CD64/CD28/z) since the latter was the best construct in inducing a satisfactory level of T cell trasduction. The engineered T cells rapidly expanded, in vitro, reaching an average of T cell transduction up to 60% on the third week of culture. CD64-CR,CD8 T cells mainly showed a central memory phenotype. Importantly, CD64-CR T cells sensed either B7-H3 positive colorectal carcinoma cell lines (HCT-116 and HT-29) or head and neck carcinoma cells (FaDu and A253) or a variety of additional cell lines (shown in Table 3). Following conjugation with cancer cells, CD64-CR triggered a powerful HLA-unrestricetd T cell-mediated cytotoxicity leading to a strong cancer cell elimination, in vitro, even at very low level of E:T cell ratio. These data evidenced that CD64-CR recognized unknown cell surface ligand(s) on cancer cells. CD64-CR T cells preferentially recognized cancer cells since marginally affected the viability of skin fibroblasts and myoblast and to a lesser extent lung fibroblasts, particularly when utilized at low E:T ratio. Confocal microscopy studies strongly suggested that CD64-CR T cells, used at least the CD64-CR T cell killing machiney to exert their cytotoxic effects. This is supported by laboratory data in which CD64-CR polarized and cololocalized with perforine, in the presence of cancer cells. Furthermore, the oponization of the cell surface of the cancer cells with an anti-B7-H3, 376.96 or cetuximab mAb resulted in a strong enhancement of the CD64-CR T cell-mediated cytotoxicity. Thus, CD64-CR confers T cells with a NK like cytotoxic function involving both an endogenous antibody independent cytotoxicity and ADCC which was triggered by the anti-B7-H3 mAb. The cytotoxic potential of CD64-CR T was evaluated by a direct comparison with that of the CD16-CR or a conventional B7-H3-CAR. CD64-CR T cells and B7-H3-CAR T cells mediated an equivalent extent of cytotoxicity on all cancer cells utilized. In contrast, CD16-CR T cells killed only in the presence of cetuximab. Overall, CD64-CR T cells mediate an NK like activity consisting of an HLA-unrestricted cellular cytotoxicity, which is optimized by the use of the 376.96 mAb. These cells preferentially killed cancer cells. As a result, such a combination could be used to develop a rational cell-based immunotherapy of B7-H3 positive and negative solid and hematopoietic cancer cells.

### Chimeric Receptors (CRs)

In one embodiment, the present invention relates to a chimeric receptor (CR). The CR includes an extracellular domain, a transmembrane domain, and an intracellular domain. The extracellular domain includes a target-specific binding element otherwise referred to as a binding domain. In certain embodiments, the intracellular domain or otherwise the cytoplasmic domain includes, a costimulatory signaling region and a zeta chain portion. The costimulatory signaling region refers to a portion of the CR including the intracellular domain of a costimulatory molecule. Costimulatory molecules are cell surface molecules other than antigen receptors or their ligands that are required for an efficient response of lymphocytes.

Between the extracellular domain and the transmembrane domain of the CR, or between the cytoplasmic domain and the transmembrane domain of the CR, there may be optionally incorporated a spacer domain. As used herein, the term “spacer domain” generally means any oligo- or polypeptide that functions to link the transmembrane domain to, either the extracellular domain or, the cytoplasmic domain in the polypeptide chain. A spacer domain may include up to 300 amino acids, preferably 10 to 100 amino acids and most preferably 25 to 50 amino acids.

The extracellular domain, transmembrane domain, and intracellular domain can be derived from any desired source of such domains.

In one embodiment, the CR includes an amino acid sequence as follows:

(MS-SP)-CD64.28.ζ
(SEQ ID NO: 1)
MGILTSFGDDMWLLTTLLLWVPVGGQVDTTKAVITLQPPWVSVFQEETVT
LHCEVLHLPGSSSTQWFLNGTATQTSTPSYRITSASVNDSGEYRCQRGLS
GRSDPIQLEIHRGWLLLQVSSRVFTEGEPLALRCHAWKDKLVYNVLYYRN
GKAFKFFHWNSNLTILKTNISHNGTYHCSGMGKHRYTSAGISVTVKELFP
APVLNASVTSPLLEGNLVTLSCETKLLLQRPGLQLYFSFYMGSKTLRGRN
TSSEYQILTARREDSGLYWCEAATEDGNVLKRSPELELQVLGLQLPTPVW
FHPGPSKPFWVLVVVGGVLACYSLLVTVAFIIFWVRSKRSRLLHSDYMNM
TPRRPGPTRKHYQPYAPPRDFAAYRSVDRVKFSRSADAPAYQQGQNQLYN
ELNLGRREEYDVLDKRRGRDPEMGGKPRRKNPQEGLYNELQKDKMAEAYS
EIGMKGERRRGKGHDGLYQGLSTATKDTYDALHMQALPPR

In another embodiment, the CR includes an amino acid sequence as follows:

CD64.28.ζ
(SEQ ID NO: 8)
MGFLTTLLLWVPVDGQVDTTKAVITLQPPWVSVFQEETVTLHCEVLHLPG
SSSTQWFLNGTATQTSTPSYRITSASVNDSGEYRCQRGLSGRSDPIQLEI
HRGWLLLQVSSRVFTEGEPLALRCHAWKDKLVYNVLYYRNGKAFKFFHWN
SNLTILKTNISHNGTYHCSGMGKHRYTSAGISVTVKELFPAPVLNASVTS
PLLEGNLVTLSCETKLLLQRPGLQLYFSFYMGSKTLRGRNTSSEYQILTA
RREDSGLYWCEAATEDGNVLKRSPELELQVLGLQLPTPVWFHPGPSKPFW
VLVVVGGVLACYSLLVTVAFIIFWVRSKRSRLLHSDYMNMTPRRPGPTRK
HYQPYAPPRDFAAYRSVDRVKFSRSADAPAYQQGQNQLYNELNLGRREEY
DVLDKRRGRDPEMGGKPRRKNPQEGLYNELQKDKMAEAYSEIGMKGERRR
GKGHDGLYQGLSTATKDTYDALHMQALPPR*

In yet another embodiment, the CR includes an amino acid sequence as follow:

CD64(EC-TM).28.ζ
(SEQ ID NO: 15)
MGFLTTLLLWVPVDGQVDTTKAVITLQPPWVSVFQEETVTLHCEVLHLPG
SSSTQWFLNGTATQTSTPSYRITSASVNDSGEYRCQRGLSGRSDPIQLEI
HRGWLLLQVSSRVFTEGEPLALRCHAWKDKLVYNVLYYRNGKAFKFFHWN
SNLTILKTNISHNGTYHCSGMGKHRYTSAGISVTVKELFPAPVLNASVTS
PLLEGNLVTLSCETKLLLQRPGLQLYFSFYMGSKTLRGRNTSSEYQILTA
RREDSGLYWCEAATEDGNVLKRSPELELQVLGLQLPTPVWFHVLFYLAVG
IMFLVNTVLWVTIVRSKRSRLLHSDYMNMTPRRPGPTRKHYQPYAPPRDF
AAYRSVDRVKFSRSAEPPAYQQGQNQLYNELNLGRREEYDVLDKRRGRDP
EMGGKPRRKNPQEGLYNELQKDKMAEAYSEIGMKGERRRGKGHDGLYQGL
STATKDTYDALHMQALPPR

### Leader Sequence

In one embodiment, the CR includes a leader sequence. Exemplary leader sequences include a mouse CD64 or human CD64 leader sequences.

The mouse CD64 leader sequence, for example, includes the following amino acids:

(SEQ ID NO: 2)
MGILTSFGDDMWLLTTLLLWVPVGG

The CD64 human leader sequence, for example, includes the following amino a

(SEQ ID NO: 9)
MGFLTTLLLWVPVDG

### Binding Domain

The binding domain may be obtained from any of the wide variety of extracellular domains or secreted proteins associated with ligand binding and/or signal transduction.

The present invention, in one embodiment, includes a binding domain of CD64. CD64 is a type of integral membrane glycoprotein known as an Fc receptor that binds monomeric IgG-type antibodies with high affinity. It is also known as Fc-gamma receptor (FcγRI). After binding IgG, CD64 interacts with an accessory chain known as the common γ chain (γ chain), which possesses an ITAM motif that is necessary for triggering cellular activation.

Structurally CD64 is composed of a signal peptide that allows its transport to the surface of a cell, three extracellular immunoglobulin domains of the C2-type that it uses to bind antibody, a hydrophobic transmembrane domain, and a short cytoplasmic tail. CD64 is the FcγR with high-affinity for monomeric IgGs and, unlike other members, it is composed of three extracellular Ig-like domains (D1, D2, and D3). CD64 is expressed on monocytes/macrophages and dendritic cells (DCs) while neutrophils and mast cells express CD64 after stimulation with interferon-γ (IFNγ) or granulocyte colony-stimulating factor (G-CSF). The engagement of CD64 on effector cells results in ADCC and antibody-dependent cellular phagocytosis (ADCP) of target cells.

The present invention includes a binding domain that binds that binds monomeric IgG-type antibodies with high affinity. The CR of the invention can be engineered to include any CD moiety that is specific to binding antibodies. The binding domain can be any domain that binds to any antibody including including but not limited to monomeric IgG-type antibodies, monoclonal antibodies, polyclonal antibodies, synthetic antibodies, scFvs, human antibodies, humanized antibodies, and fragments thereof.

In one embodiment, the CD64 binding domain includes the following amino acids:

(SEQ ID NO: 3)
QVDTTKAVITLQPPWVSVFQEETVTLHCEVLHLPGSSSTQWFLNGTATQT
STPSYRITSASVNDSGEYRCQRGLSGRSDPIQLEIHRGWLLLQVSSRVFT
EGEPLALRCHAWKDKLVYNVLYYRNGKAFKFFHWNSNLTILKTNISHNGT
YHCSGMGKHRYTSAGISVTVKELFPAPVLNASVTSPLLEGNLVTLSCETK
LLLQRPGLQLYFSFYMGSKTLRGRNTSSEYQILTARREDSGLYWCEAATE
DGNVLKRSPELELQVLGLQLPTPVWFH

Other binding domain regions useful for engineering CD64 CRs include those found at the interface of FcγRI with the lower hinge Fc region (Lu et al., PNAS 112: 823-838, 2015). The interface between the receptor and the two Fc chains can be divided into three regions: between the FcγRI D2 domain C-strand (Tyr-133-Leu-136), C′-strand (Lys-142-His-148), and C′E loop (His-148-Trp-149) and the lower hinge region from the Fc A-chain; between the receptor D1-D2 interdomain hinge (Arg-102-Trp-104), D2 domain BC loop (Trp-127-Tyr-133), and lower hinge from the Fc B-chain; and between the FcγRI D2 domain FG loop (Met-171-Tyr-176) and the Fc glycans. Accordingly, useful CD64-CR receptors typically include one or more of the FcγRI D2 domain C-strand (Tyr-133-Leu-136), C′-strand (Lys-142-His-148), and C′E loop (His-148-Trp-149). In some examples, the numbering of these amino acid sequences is relative to the numbering of the exemplary CD64 amino acid sequence set forth in Uniprot Accession No. P12314-1, reproduced below:

| (SEQ ID NO: 21) | | | | |
|---|---|---|---|---|
| 10 | 20 | 30 | 40 | 50 |
| MWFLTTLLLW | VPVDGQVDTT | KAVITLQPPW | VSVFQEETVT | LHCEVLHLPG |
| 60 | 70 | 80 | 90 | 100 |
| SSSTQWELNG | TATQTSTPSY | RITSASVNDS | GEYRCQRGLS | GRSDPIQLEI |
| 110 | 120 | 130 | 140 | 150 |
| HRGWLLLQVS | SRVFTEGEPL | ALRCHAWKDK | LVYNVLYYRN | GKAFKFFHWN |
| 160 | 170 | 180 | 190 | 200 |
| SNLTILKTNI | SHNGTYHCSG | MGKHRYTSAG | ISVTVKELFP | APVLNASVTS |
| 210 | 220 | 230 | 240 | 250 |
| PLLEGNLVTL | SCETKLLLQR | PGLQLYFSFY | MGSKTLRGRN | TSSEYQILTA |
| 260 | 270 | 280 | 290 | 300 |
| RREDSGLYWC | EAATEDGNVL | KRSPELELQV | LGLQLPTPVW | FHVLFYLAVG |
| 310 | 320 | 330 | 340 | 350 |
| IMFLVNTVLW | VTIRKELKRK | KKWDLEISLD | SGHEKKVISS | LQEDRHLEEE |
| 360 | 370 | | | |
| LKCQEQKEEQ | LQEGVHRKEP | QGAT. | | |

### Transmembrane Domain

With respect to the transmembrane domain, the CR can be designed to include a transmembrane domain that is fused to the extracellular domain of the CR. In one embodiment, the transmembrane domain that naturally is associated with one of the domains of CD64 in the CR is used. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. include at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD 154. Alternatively, the transmembrane domain may be synthetic, in which case it will include predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CR. A glycine-serine doublet provides a particularly suitable linker.

In one embodiment, the transmembrane domain, for example, includes the following amino acids of the CD28 transmembrane domain:

(SEQ ID NO: 5)
FWVLVVVGGVLACYSLLVTVAFIIFWV

In one embodiment, the transmembrane domain, for example, includes the CD64 transmembrane domain:

(SEQ ID NO: 18)
VLFYLAVGIMFLVNTVLWVTI

In one embodiment, the transmembrane domain, for example, includes the CD64 transmembrane domain with a valine:

(SEQ ID NO: 22)
VLFYLAVGIMFLVNTVLWVTIV

### Intracellular Domain

The cytoplasmic domain or otherwise the intracellular domain of the CR of the invention is responsible for activation of at least one of the normal effector functions of the immune cell in which the CR has been placed in. The term “effector function” refers to a specialized function of a cell. Effector function of a T cell, for example, may be cytolytic activity or helper activity including the secretion of cytokines. Thus, the term “intracellular signaling domain” refers to the portion of a protein which transduces the effector function signal and directs the cell to perform a specialized function. While usually the entire intracellular domain can be employed, in many cases it is not necessary to use the entire chain. To the extent that a truncated portion of the intracellular domain is used, such truncated portion may be used in place of the intact chain as long as it transduces the effector function signal. The term intracellular domain is thus meant to include any truncated portion of the intracellular domain sufficient to transduce the effector function signal.

Preferred examples of intracellular domains for use in the CR of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of intracellular signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary intracellular signaling sequences regulate primary activation of the TCR complex either in a stimulatory way, or in an inhibitory way. Primary intracellular signaling sequences that act in a stimulatory manner may contain signaling motifs which are known as immunoreceptor tyrosine-based activation motifs or ITAMs.

Examples of ITAM containing primary intracellular signaling sequences that are of particular use in the invention include those derived from TCR zeta, FcR gamma, FcR beta, CD3 gamma, CD3 delta, CD3 epsilon, CD5, CD22, CD79a, CD79b, and CD66d. It is particularly preferred that intracellular signaling molecule in the CR of the invention includes an intracellular signaling sequence derived from CD3 zeta.

In a preferred embodiment, the intracellular domain of the CR can be designed to include the CD3-zeta signaling domain by itself or combined with any other desired cytoplasmic domain(s) useful in the context of the CR of the invention. For example, the intracellular domain of the CR includes a CD3 zeta chain portion and a costimulatory signaling region. The costimulatory signaling region refers to a portion of the CR including the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1 BB (CD137), OX40, CD30, CD40, 4-1 BB, PD-1, ICOS, lymphocyte function-associated antigen-1 (LFA-1), CD2, CD7, LIGHT, NKG2C, B7-H3, and a ligand that specifically binds with CD83, and the like. Thus, while the invention in exemplified primarily with CD28 as the co-stimulatory signaling element, other costimulatory elements are within the scope of the invention.

The intracellular signaling sequences within the intracellular domain of the CR of the invention may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the intracellular domain is designed to include the signaling domain of CD3-zeta and the signaling domain of CD28.

CD3-zeta signaling domain
(SEQ ID NO: 7)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.
CD28 signaling domain
In one embodiment, the intracellular domain
include the amino acid sequence
(SEQ ID NO: 13)
RSKRSRLLHSDYMNMTPRRPGPTRKHYQPYAPPRDFAAYRS

In other embodiments, the intracellular domain, for example, includes CD28 and CD3-zeta signaling domains.

(SEQ ID NO: 23)
CD28 and CD3-zeta signaling domains
RSKRSRLLHSDYMNMTPRRPGPTRKHYQPYAPPRDFAAYRSVDRVKFSR
SAEPPAYQQGQNQLYNELNLGRREEYDVLDKRRGRDPEMGGKPRRKNPQ
EGLYNELQKDKMAEAYSEIGMKGERRRGKGHDGLYQGLSTATKDTYDAL
HMQALPPR

### Sources of Immune Cells

In certain embodiments, prior to expansion, a source of immune cells (e.g., a T cell or other immune cell described herein) is obtained from a subject. Non-limiting examples of subjects include humans, dogs, cats, mice, rats, and transgenic species thereof. Preferably, the subject is a human. T cells can be obtained from a number of sources, including peripheral blood mononuclear cells, bone marrow, lymph node tissue, spleen tissue, umbilical cord, and tumors. In certain embodiments, any number of T cell lines available in the art, may be used. In certain embodiments, T cells can be obtained from a unit of blood collected from a subject using any number of techniques known to the skilled artisan, such as Ficoll separation. In one embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood by lysing the red blood cells and depleting the monocytes, for example, by centrifugation through a

PERCOLL™ gradient. Alternatively, T cells can be isolated from umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19 and CD56.

Depletion of these cells can be accomplished using an isolated antibody, a biological sample including an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CDI Ib, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/nil is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, 01100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells include the population of T cells. In yet another embodiment, purified T cells include the population of T cells.

### Cell Expansion

In certain embodiments, the immune cells (e.g., a T cell or other immune cell) disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the T cells expand in the range of about 20 fold to about 50 fold.

Following culturing, the T cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The T cell medium may be replaced during the culture of the T cells at any time. Preferably, the T cell medium is replaced about every 2 to 3 days. The T cells are then harvested from the culture apparatus whereupon the T cells can be used immediately or cryopreserved to be stored for use at a later time. In one embodiment, the invention includes cryopreserving the expanded T cells. The cryopreserved T cells are thawed prior to introducing nucleic acids into the T cell.

In another embodiment, the method includes isolating T cells and expanding the T cells. In another embodiment, the invention further includes cryopreserving the T cells prior to expansion. In yet another embodiment, the cryopreserved T cells are thawed for electroporation with the RNA encoding the chimeric membrane protein.

Procedures for ex vivo expansion and culture of cells are known in the art.

Further a medium used to culture the T cells may include an agent that can co-stimulate the T cells. For example, an agent that can stimulate CD3 is an antibody to CD3, and an agent that can stimulate CD28 is an antibody to CD28.

### Pharmaceutical Compositions

Pharmaceutical compositions of the present invention may include the modified immune cell (e.g., a T cell or other immune cell) as described herein, in combination with one or more pharmaceutically or physiologically acceptable carriers, diluents or excipients. Such compositions may include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

The cells of the invention to be administered may be autologous, allogeneic or xenogeneic with respect to the subject undergoing therapy.

Cells of the invention can be administered in dosages and routes and at times to be determined in appropriate pre-clinical and clinical experimentation and trials. Cell compositions may be administered multiple times at dosages within these ranges.

Administration of the cells of the invention may be combined with other methods useful to treat the desired disease or condition as determined by those of skill in the art.

It can generally be stated that a pharmaceutical composition including the modified immune cells (e.g., a T cell or other immune cell) described herein may be administered at a dosage of 10⁴ to 10⁹ cells/kg body weight, in some instances 10⁵ to 10⁶ cells/kg body weight, including all integer values within those ranges. T cell compositions, for example, may also be administered multiple times at these dosages. The cells can be administered by using infusion techniques that are commonly known in immunotherapy (see, e.g., Rosenberg et al., New Eng. J. of Med. 319: 1676, 1988). The optimal dosage and treatment regime for a particular patient can readily be determined by one skilled in the art of medicine by monitoring the patient for signs of disease and adjusting the treatment accordingly.

The administration of the modified T cells, for example, may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. These techniques are applicable to the production of the polynucleotides and polypeptides of the invention, and, as such, may be considered in making and practicing the invention. Particularly useful techniques for particular embodiments will be discussed in the sections that follow.

### Therapy

Host cells (e.g., immune cells) expressing CRs (the encoding nucleic acids or vectors including such) described herein are useful for enhancing ADCC or ADCP in a subject and/or for enhancing the efficacy of an antibody-based immunotherapy. In some embodiments, the subject is a mammal, such as a human. In some embodiments, the subject is a human cancer patient. In some embodiments, the subject has been treated or is being treated with any of the therapeutic antibodies described herein.

The immune cells are mixed with a pharmaceutically acceptable carrier to form a pharmaceutical composition.

Typically, an effective amount of the immune cells expressing any of the CR constructs described herein are administered into a subject in need of the treatment. The immune cells may be autologous to the subject, i.e., the immune cells are obtained from the subject in need of the treatment, genetically engineered for expression of the CR constructs, and then administered to the same subject. Alternatively, the host cells are allogeneic cells, i.e., the cells are obtained from a first subject, genetically engineered for expression of the CR construct, and administered to a second subject that is different from the first subject but of the same species. For example, allogeneic immune cells may be derived from a human donor and administered to a human recipient who is different from the donor.

In some embodiments, the immune cells are administered to a subject in an amount effective in enhancing ADCC activity by least 10% or 20%, e.g., 30%, 40%, 50%, 60%, 70%, 80%, 90%, 100%, 2-fold, 5-fold, 10-fold, 20-fold, 50-fold, 100-fold or more.

In some embodiments, the immune cells are co-administered with a therapeutic Fc-containing therapeutic agent (e.g., an antibody or Fc fusion molecule such as Fc fusion protein) to enhance the efficacy of the immunotherapy.

In any of the methods described herein, the immune cells such as the T lymphocytes are autologous cells isolated from the subject who is subject to the treatment. In one specific embodiment, prior to re-introduction into the subject, the autologous immune cells (e.g., T lymphocytes) are activated and/or expanded ex vivo. In another embodiment, the immune cells (e.g., T lymphocytes or NK cells) are allogeneic cells.

The CRs of the disclosure may be used for treatment of any cancer, including, without limitation, those for which a specific antibody with an Fc portion that binds to the Fc binder in the CR exists or is capable of being generated.

Typically, an effective amount of the immune cells expressing CRs, Fc-containing therapeutic agents (e.g., Fc-containing therapeutic proteins such as Fc fusion proteins and therapeutic antibodies), or compositions thereof is administered to a subject (e.g., a human cancer patient) in need of the treatment via a suitable route, such as intravenous administration. Any of the immune cells expressing CRs, Fc-containing therapeutic agents, or compositions thereof may be administered to a subject in an effective amount. As used herein, an effective amount refers to the amount of the respective agent (e.g., the host cells expressing CRs, Fc-containing therapeutic agents, or compositions thereof) that upon administration confers a therapeutic effect on the subject. Determination of whether an amount of the cells or compositions described herein achieved the therapeutic effect is apparent to one of skill in the art.

In some embodiments, the subject is a human cancer patient. For example, the subject are a human patient suffering from carcinoma, lymphoma, sarcoma, blastoma, or leukemia. Examples of cancers for which administration of the cells and compositions disclosed herein may be suitable include, for example, a wide range of hematologic and solid B7-H3 positive tumors (such as colorectal cancer, breast cancer, non-small cell lung cancer, head and neck cancer, glioblastoma, leukemia, myeloma).

In accordance with the present disclosure, patients are treated by infusing therapeutically effective doses of immune cells such as T lymphocytes including a CR described. Infusions are repeated as until the desired response is achieved.

In some embodiments, the immune cells expressing any of the CRs disclosed herein are administered to a subject who has been treated or is being treated with an Fe-containing therapeutic agent (e.g., an Fc-fusion protein or a therapeutic antibody). The immune cells expressing any one of the CRs disclosed herein may be co-administered with an Fe-containing therapeutic agent. For example, the immune cells may be administered to a human subject simultaneously with a therapeutic antibody.

Alternatively, the immune cells may be administered to a human subject during the course of an antibody-based immunotherapy.

Examples of therapeutic Fe-containing therapeutic agent include, without limitation, any antibody that binds a cell surface antigen, e.g., cetuximab, enoblituzumab, or other therapeutic antibody used in cancer treatment (for example, rituximab, HERCEPTIN® (trastuzumab), daratumumab, mogamolizumab, panitumumab, and atezolizumab).

The administration of a Fe-containing therapeutic agent is performed by any suitable route, including systemic administration as well as administration directly to the site of the disease (e.g., to primary tumor).

The compositions and methods described in the present disclosure may be utilized in conjunction with other types of therapy for cancer, such as chemotherapy, surgery, radiation, gene therapy, and so forth. Such therapies are administered simultaneously or sequentially (in any order) with the immunotherapy according to the present disclosure.

### EXAMPLES

### Example 1—the Efficient Expression of Human CD64-Chimeric Receptor (CR) in T Lymphocytes Required Mouse CD64 Signal Peptide

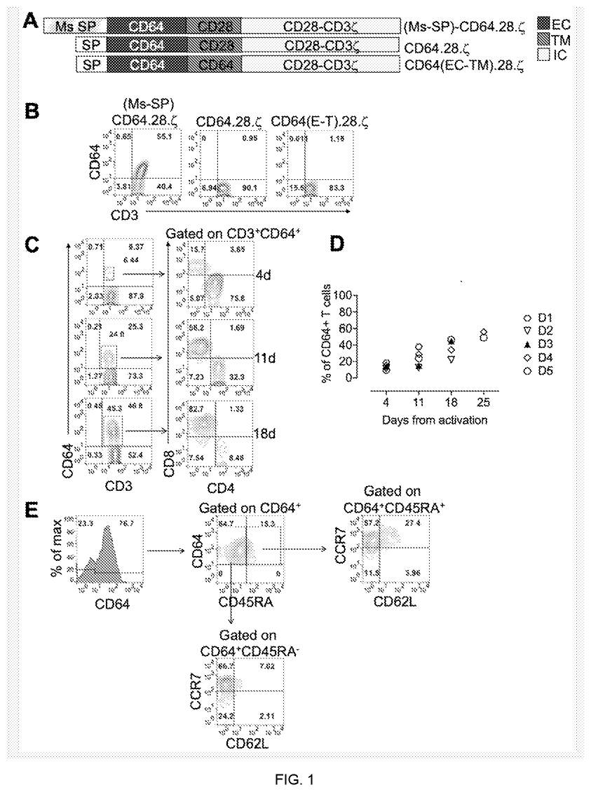

Four different CD64 chimeric receptors (CRs) were designed and generated (FIG. 1A). The (Ms-SP)-CD64.28.ζ and CD64.28.ζ constructs had identical sequences with the only difference in the signal peptide (SP): the former contained the SP derived from Mus musculus CD64 protein, the latter had the SP of the human CD64 molecule. They shared the transmembrane (TM) and intracellular (IC) domains derived from the CD28 molecule and the cytoplasmic region of the CD3 chain. The third and the fourth constructs consisted of the SP, the EC, and the TM regions of the human CD64 linked to the CD28 and CD3ζ IC signaling domains through two different transmembrane domains: in the CD64(EC-TM).28.ζ chimeric receptor the TM domain was from CD64 while in the CD64.8.28.ζ (not shown) was from CD8α.

The four CD64 chimeric receptor variants were cloned into retroviral vectors and transduced in human T cells. After transduction, the expression of the chimeric receptors was tested by flow cytometry (FIG. 1B). Only the (Ms-SP)-CD64.28.ζ construct (hereafter referred as CD64-CR), containing the signal peptide of the murine CD64, was efficiently expressed in CD3+ T cells. Otherwise, the remaining three CRs with the human CD64 SP were not expressed (FIG. 1B). The expression of the CD64-CR in CD3+ T cells was measured once a week at 4-, 11- and 18-day post-transduction and a preferential expansion of the CD3+CD64+ cells was observed over time (FIG. 1C, left panels). From the 6.44% of CD64+CD3+ cells detected at day 4 post-transduction, the percentage of CD64+ cells reached 45.3% at 18-day post transduction. A cumulative analysis of the time-dependent expansion of CD64+CD3+ cells in five different donors is showed in FIG. 1D.

The distribution of the CD64-CR on the CD4+ and CD8+ T cell subsets was also analyzed. As expected, the CD4/CD8 T cell ratio was inverted during in vitro culture. Four days after transduction, the CD64+CD4+ cell proportion was higher than that of CD64+CD8+ cells (75.6% vs 15.7% respectively) while at 18 days from transduction the percentage of CD64+CD8+ overcame the percentage of CD64+CD4+ cells (82.7% vs 8.48% respectively) (FIG. 1C, right panels).

The phenotypic analysis, performed by flow cytometry at 30 days after activation, revealed that CD64+ T cells contained 27.4% of stem cell memory (CD45RA+CCR7+CD62L+), 7.02% of central memory (CD45RA−CCR7+CD62L+), 24.2% of effector memory (CD45RA−CCR7−CD62L−), and 11.5% of effector T cells (CD45RA+CCR7−CD62L−) (FIG. 1E).

### Example 2—CD64-CR T Cell Mediated the Direct Elimination of CRC Cell Lines Independent of Antibody Ligation

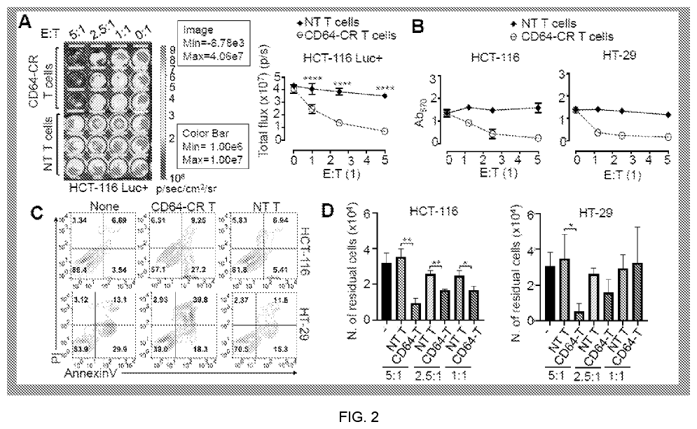

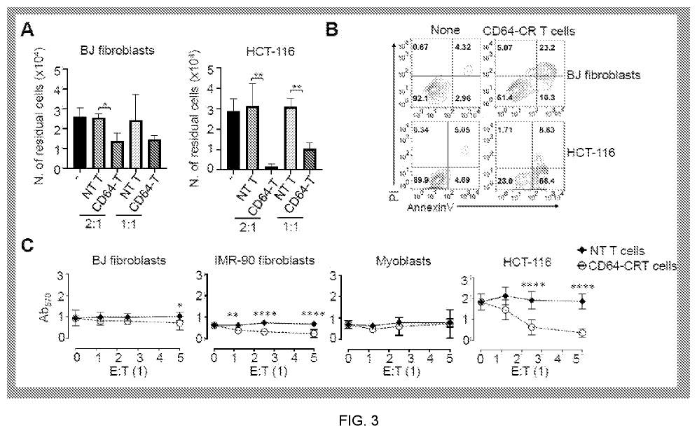

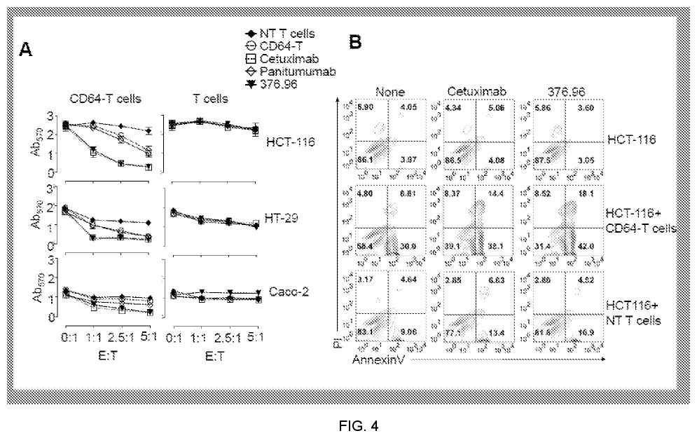

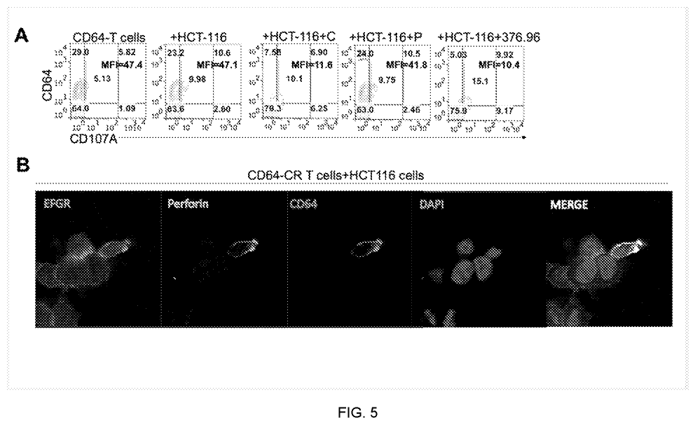

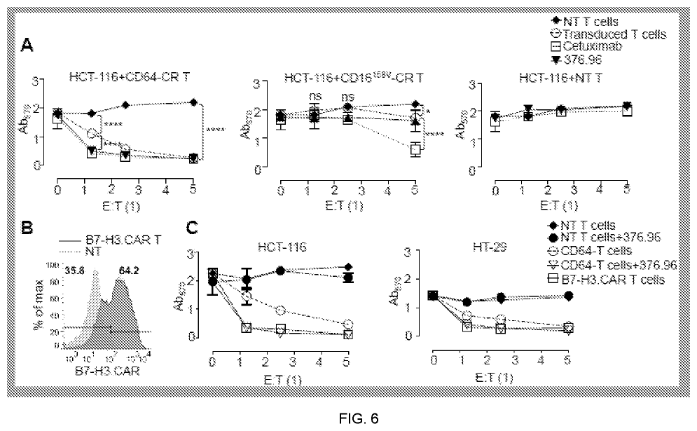

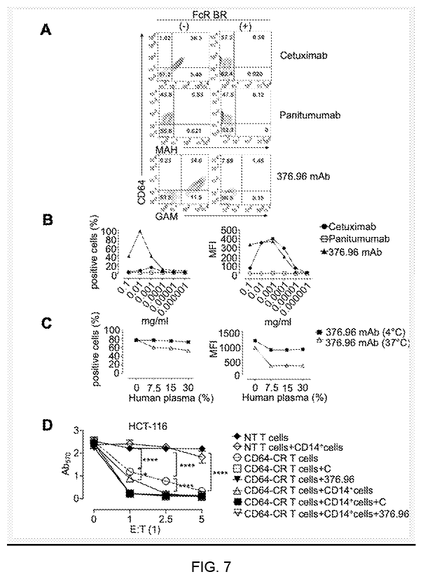

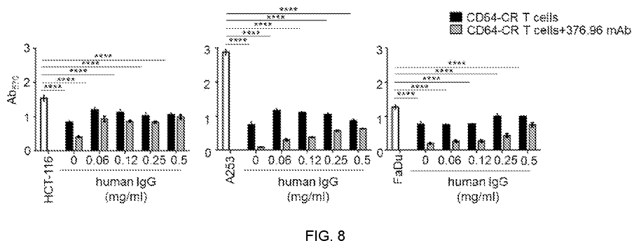

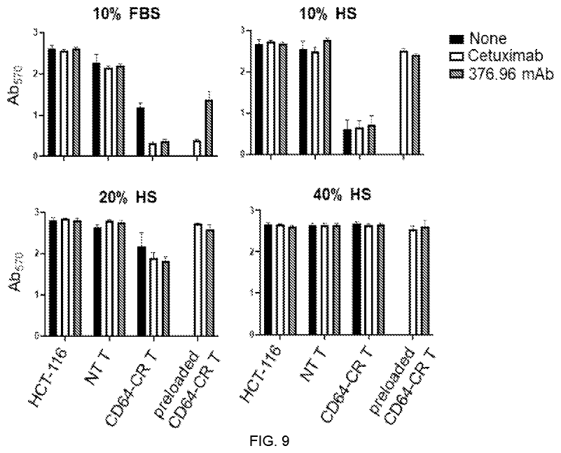

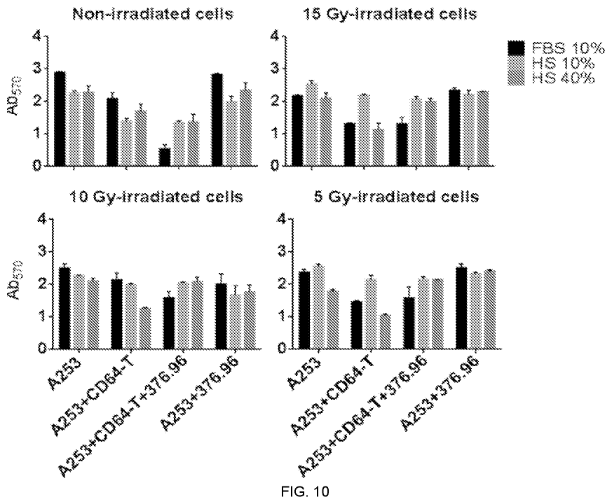

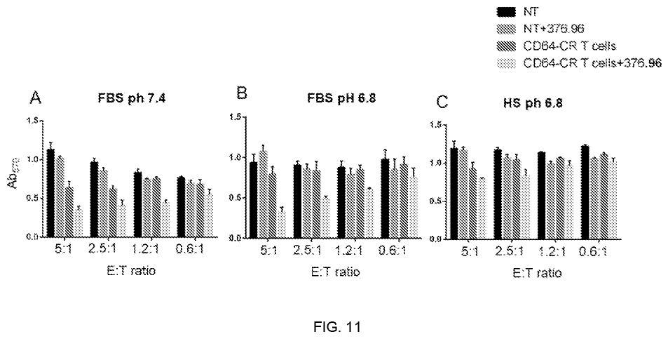

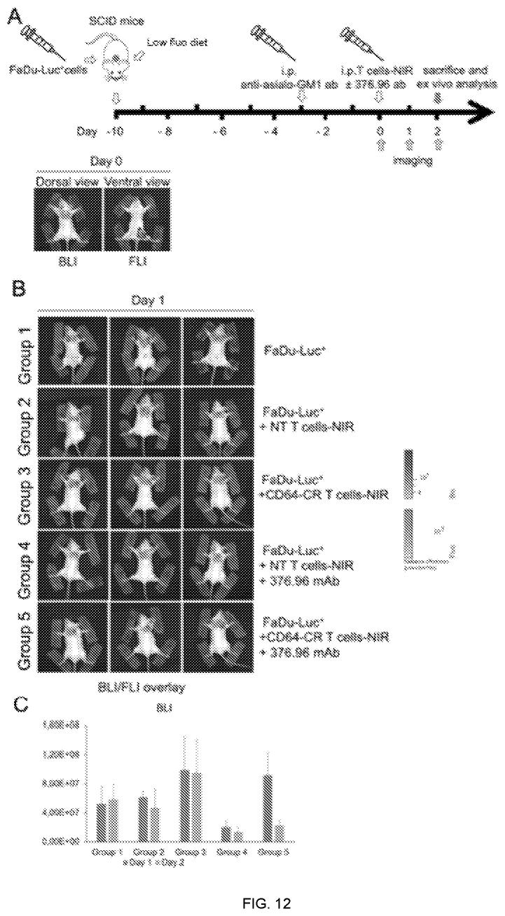

The high-affinity FcγRI/CD64 receptor is broadly known for its role in mediating ADCC and ADCP by monocytes/macrophages upon mAb binding. Here, we described an alternative ability of the CD64-CR consisting of a direct tumor target cell recognition and elimination, without the need of an antibody link. HCT-116 Luc+, expressing the firefly luciferase protein, were co-cultured at 37° C. with CD64-CR transduced and control non transduced (NT) T cells at different E:T ratios (FIG. 2A). After 72-hour incubation, D-luciferin was added and the viability of HCT-116 Luc+ was visualized by bioimaging (FIG. 2A, left panel). The quantification of photons emitted (total flux) by HCT-116 Luc+ indicated a significant reduction of viable target cells cocultured with CD64-CR T cells than that incubated with NT T cells (FIG. 2A, right panel). Strictly similar results were obtained by the MTT assay, that confirmed a significant viability reduction of HCT-116 incubated with CD64-CR T cells and showed that this effect was not restricted to HCT-116 but it was also proved in a second CRC cell line HT-29 (FIG. 2B). This observation raised the question if the CD64-CR-driven anti-tumor effect was the result of an anti-proliferative activity or a target-cell killing mechanism. To investigate the process underlying the CD64-CR T cell direct effect, HCT-116 and HT-29 cell lines were cocultured for 4 hours with or without CD64-CR transduced or NT T cells; following the apoptosis and necrosis levels were analyzed by flow cytometry. As shown in FIG. 2C, the CD64-CR T cells clearly damaged CRC cell lines by inducing HCT-116 and HT-29 cell death. Furthermore, this data was corroborated by counting the number of HCT-116 and HT-29 remaining cells after an overnight-incubation with CD64-CR T effector cells.

The cell count experiments showed a significant elimination of HCT-116 and, to a lesser extent, HT29 cells after coculture with CD64-CR T cells, indicating, in line with flow cytometry cytotoxicity data, that CD64-CR T cells killed CRC target cells.

### Example 3—CD64-CR T Cells Showed Limited Toxic Effect on Human Normal Cells In Vitro

The direct damage induced on CRC cell lines by CD64-CR T cells suggests the presence of an unknown ligand on sensitive tumor cells. Thus, it is critical to determine whether non-tumor normal cells could be affected by CD64-CR T cells. We tested the potential toxicity of CD64-CR T lymphocytes on normal human skin fibroblast BJ, lung fibroblast IMR-90 cell lines and human primary myoblasts in vitro (FIG. 3). Firstly, BJ cells were cocultured with CD64-CR transduced and NT T cells at 2:1 and 1:1 E:T ratios (FIG. 3A). HCT-116 were cultured in the same conditions as a positive control. After 18-hour incubation, residual target cells were enumerated and a BJ cell number reduction was observed at 2:1 E:T ratio. No significant BJ cell number decrease was detected at 1:1 E:T ratio. This effect was very limited compared to the massive simultaneous elimination of HCT-116 cells by CD64-CR T cells showed both at 2:1 and 1:1 E:T ratios. Similar results were obtained in the cytotoxicity assay performed by flow cytometry (FIG. 3B). BJ fibroblasts or HCT-116 cells were cocultured at 37° C. with CD64-CR T cells at 2:1 E:T ratio and after 4-hour incubation only the 10.3% of BJ cells were annexin V+ and the 23.2% were positive for apoptotic and necrotic markers. Conversely, HCT-116 cells were drastically damaged after incubation with CD64-CR T cells, with the 66.4% of cells annexin V+ and 8.83% annexin V+ and PI+. These data were also confirmed when coculture between BJ and HCT-116 target cells and CD64-CR effector cells was prolonged for 72 hours (FIG. 3C). In the MTT assay, the CD64-CR T cell potential toxicity was also tested towards IMR-90 fibroblasts and human myoblasts. As shown in FIG. 3C, CD64-CR T cells strongly impaired HCT-116 growth, moderately affected BJ and IMR-90 cell viability while did not have any detectable effects on myoblast culture.

### Example 4—the Cytotoxic Effect of CD64-CR T Cells Against CRC Cells is Enhanced by the Addition of Cetuximab or 376.96 Anti-B7-H3 mAb

The ability of CD64-CR T cells to mediate ADCC against CRC cell lines was investigated. The Fc mAb binding assay revealed that CD64-CR specifically bound the Fc fragment of anti-EGFR IgG1 cetuximab and anti-B7-H3 IgG2A 376.96 at 4° C. (FIG. 7A-B). Conversely, CD64-CR did not bind anti-EGFR IgG2 panitumumab. Furthermore, the ligation of cetuximab and 376.96 mAb to CD64-CR was specific because it was completely abrogated in the presence of FcR BR (FIG. 7A). Dose-response experiments clearly indicated that 376.96 mAb bound to CD64-CR T cells, in term of percentage of binding and MFI, better than Cetuximab and Panitumumab. To be noted that Panitumumab did not bind at all to CD64-CR T cells. The 376.96 mAb maximum percentage of binding to CD64-CR T cells was reached at 10 ug/ml while the highest level of MFI ranged between 100-1 μg/ml of (FIG. 7B).

As known, the CD64 is the only FcγR that binds monomeric immunoglobulins (Ig) with high affinity. For that reason, we tested the possibility that free serum IgGs could saturate the CD64-CR, thus hampering the binding of therapeutic mAbs. Therefore, the binding of 376.96 mAb to CD64-CR was tested in the presence of increasing concentration of heat-inactivated human plasma both at 4° C. and 37° C. (FIG. S1C). The CD64-CR still bound the Fc fragment of 376.96 mAb in the presence of the highest concentration of 30% of human plasma at 4° C.: no differences in percentage of positive cells was found, while a slight decrease in mean fluorescence intensity was detected after plasma addition. Simultaneously, the binding assay was carried out at a more physiological temperature of 37° C. Although a reduction of the 376.96 mAb binding to the CD64-CR (both % of positive cells and MFI) was observed in the presence of different percentages of plasma, the binding was still maintained (FIG. 7C)

To determine whether the CD64-CR ability of mAb binding could be associated with a functional activity as ADCC, the CD64-transduced T cells were cocultured with EGFR+B7-H3+HCT-116, HT-29 and Caco-2 target cells in the presence of cetuximab, panitumumab or 376.96. After 72-hours incubation, we found that CD64-CR both directly impaired HCT-116 and HT-29 cell viability and exerted ADCC in the presence of cetuximab or 376.96 mAb (FIG. 4A). CaCo-2 CRC cell line was resistant to the CD64-CR T cells direct elimination however their viability was significantly reduced by cetuximab- and 376.96 mAb-induced ADCC. Conversely, panitumumab did not triggered cytotoxicity against any tested CRC cell lines. Non-transduced T cells had not detectable effect on target cell viability (FIG. 4A).

Furthermore, the direct cytotoxicity and the ADCC elicited by CD64-CR T cells on CRC HCT-116 cell line was also confirmed using a flow-cytometry assay (FIG. 4B). CD64-CR transduced or NT T cells were cocultured with HCT-116 cells at an E:T ratio of 2:1 for 18 hours in the presence or absence of 1 μg/ml of cetuximab or 376.96 mAb. The percentage of apoptosis/necrosis of target cells was analyzed by annexin V and PI staining. As clearly shown in FIG. 4B, the CD64-CR T cells induced apoptosis and necrosis in HCT-116 and the percentage of damaged cells was increased by the addition of cetuximab or 376.96 mAb. No detrimental effect was observed by the NT control T cells.

### Example 5—Comparison of CD64-CR with CD16^{158V}-CR and B7-H3.CAR

In our previous study, we demonstrated the ability of CD16^{158V}-CR T cells to overcome the resistance to cetuximab of KRAS-mutated CRC HCT-116 and NSCL A549 cell lines (Arriga et al., Int J Cancer 2020; 146:2531-8). Here, we compared the efficiency of T cells containing CD64-CR with the T cells, from the same donor, expressing CD16^{158V}-CR in HCT-116 cell eliminating, with or without cetuximab or 376.96 mAb. After retroviral transduction, transduced and NT T cells were incubated with target cells for 72 hours at 37° C. FIG. 6A shows the superior anti-CRC activity of CD64-CR T cells than CD16^{158V}-CR T cells. The CD64-CR T cells reduced HCT-116 viability by two synergistic mechanisms: the former was the direct target cell elimination; the latter was the ADCC mechanism triggered both by cetuximab and anti-B7-H3 376.96 mAb. Conversely, the CD16^{158V}-CR T cells inhibited the HCT-116 cells growth only in combination with cetuximab (FIG. 6A). This data indicated that exclusively the CD64-CR was effective in inducing T cell cytotoxicity against HCT-116 cells by the crosslinking of 376.96 mAb. From this observation, we examined the anti-CRC functionality of CD64-CR T cells combined with 376.96 mAb in comparison with the classical anti-B7-H3 chimeric receptor (B7-H3.CAR). After we successfully expressed the B7-H3 CAR on T lymphocytes (FIG. 6B), the B7-H3.CAR T cells were tested against HCT-116 or HT-29 cells simultaneously to CD64-CR expressing T cells in combination with the 376.96 mAb. The CD64-CR T cells armed with the mAb 376.96 in vitro exerted similar anti-cancer activity to the classical B7-H3.CAR (FIG. 6C).

### Examples 6 and 7—Cytotoxicity

To define the mechanism underlying the cytotoxic effect exerted by CD64-CR T cells against CRC cells, we investigated the CD107A release by CD64-CR T cells, cultured with target cells, in the presence or absence of mAbs specific for cell surface TAAs. Upon conjugation with HCT-116 cells, CD64-CR T cells mobilized CD107A. Importantly, the CD107A release was enhanced in the presence of cetuximab and the 376.96 mAb. In addition, both cetuximab and 376.96 mAbs induced down-regulation of the CD64-CR. In contrast, panitumumab did not enhance the anti-CD107A mobilization and did not down-regulate the CD64-CR molecule. The 376.96 seemed to be more powerful than cetuximab in term of the extent of CD107A mobilization. Then, we explored the involvement of the CD64-CR T cell lytic machinery by evaluating the distribution of perforin in relationship with CD64-CRs in the CD64-CR T cells. HCT-116 cells were incubated with CD64-CR expressing T cells for 30 min, at 37° C., at an E:T of 2:1, We found that CD64 polarized and colocalized with perforine at the cell surface of the CD64-CR T cells suggesting that a granule-dependent cytotoxicity may be involved (FIG. 5B)

### Example 8—Direct CD64-T Cell Cytotoxicity is Antibody-Independent

FIG. 8 shows a forty-eight-hour MTT cell viability assay of HCT116 cells incubated with CD64-CR T cells in 10% FCS, supplemented with scalar concentrations of human IgGs. Cancer cell lines, as indicated in the X-axes, were incubated at 37° C., with or without CD64-T cells, at an E:T ratio of 2:1. Results indicate that, in the presence of IgG, the anti-cancer CD64-CR T cells were not affected (black bars) while ADCC mediated by the 376.96 mAb was inhibited in a dose-dependent manner (grey bars). These data strongly suggest that the direct CD64-T cell cytotoxicity is antibody-independent.

FIG. 9 shows the effects of human serum on the anti-tumor activity of CD64-T cells on HCT116 cells in the presence of a different concentrations of human serum while 10% FBS medium was utilized as a positive control. CD64-T cells or non transduced T (NT T) cells were incubated at 37° C. with HCT116 cells at an E:T (effector (E) to target (T)) ratio of 2:1 with or without 1 μg/ml of cetuximab or 376.96 mAb, in the presence of 10% of fetal bovine serum (FBS) or 10%, 20% and 40% of heat-inactivated human serum (HS). After a 96-hour incubation, effector cells were removed and the viability of the remaining target cells was assessed by the MTT assay. As a result, in the presence of 10% of HS and to a lesser extent in 20% of HS, CD64-T cells clearly affected the viability of HCT116 cells while the ADCC activity of either the 376.96 or cetuximab was abolished. Concomitant preloading of CD64-T cells with the indicated mAbs did not reverse the inhibitory effect on ADCC. In contrast, CD64-T cells in the presence of 10% of FBS mediated direct antitumor activity and ADCC. These results suggest that a high concentration of HS inhibits both CD64-T cell types of anti-tumor activity. Also, they demonstrate that the direct anti-tumor activity of CD64-T cells can be detected even in the presence of a considerable percentage of HS. Considering that the concentration of HS, at the tumor site, should be minimal, we can speculate that the anti-cancer activity of CD64-T cells can be maintained. The irradiation of cancer cells could restore the activity of CD64-T cells in terms of direct activation and ADCC was also assessed.

FIG. 10 shows that A253 cells irradiated with 15 Gy, 10 Gy, or 5 Gy or non-irradiated were plated at a density of 20,000 cells/well in a 96 well flat bottom plate. CD64-CR T cells were added to target cells at 4:1 E:T ratio in the presence of RPMI medium supplemented with 10% of FBS, 10% or 40% of HS with or without 1 μg/ml of 376.96 mAb. After incubation, effector cells were removed and target cell viability was assessed by MTT assay. The experiment shows that irradiation of tumor cells maintains the anti-tumor activity of CD64-T cells but not that of ADCC. The effect of reducing the pH of the medium to the level of the pH typically found in the tumor microenvironment was also assessed.

FIG. 11 shows results obtained utilizing the MTT assay. CD64-T cells or NT T cells were incubated for 72 h with or without HCT116 cells, at the indicated E:T ratios. ADCC was assessed in the presence of the 376.96 mAb. The pH of the media varied as follows (A) FBS 20%, ph 7.4, (B) FBS 20%, ph 6.8 (B). HS 20%, pH 6.8 (C). The results show that acidic pH does not enhance the anti-HCT116 activity of CD64-T cells which was detected only at an E:T ratio of 5:1 in the presence of 20% of HS.

### Example 10—Results of the Biodistribution of CD64-CR T Cells In Vivo

To investigate the biodistribution of CD64-CR T cells in vivo, we first injected five hundred thousand FaDu-luciferase positive (Luc+) cells subcutaneously (s.c.) into the dorsal neck of 15 CB17 SCID mice. To avoid excessive autofluorescence, mice were fed with a low fluorescence diet. Following 10 days, mice received intraperitoneal (i.p.) injections of 180 μl of saline with or without 180 μg of 376.96 mAb, followed by injection of 2.5×10⁶ T cells labeled with near-infrared cell tracker (NIR), 30 min later. On day −3, endogenous NK cells were neutralized by an i.p. injection of anti-asialo-GM1 antibody. On day 1, imaging analysis was performed and on day 2, mice were sacrificed for an ex vivo study (see FIG. 12A).

Animals were divided into 5 groups: group 1: untreated; group 2: non-transduced (NT) T cells-NIR; group 3: CD64-CR T cells-NIR; group 4: NT T cells-NIR+376.96 mAb; and group 5: CD64-CR T cells-NIR+376.96 mAb (see FIG. 12B). The in vivo distribution of T cells was monitored by fluorescence imaging (FLI) while tumor volume was visualized by bioluminescence imaging (BLI) on days 0, 1, and 2. FIG. 12B also shows BLI and FLI overlay imaging 1 day after CD64-CR or NT T cell-NIR (red signal) i.p. injection and tumor (blue signal) distribution in SCID mice groups. The data in FIGS. 12A and 12B provide evidence indicating that both NT T cells and CD64-CR T cells migrate to the tumor site. Interestingly, on day 2 of monitoring, we found that the BLI signal of cancer cells was significantly reduced in the group 5 mice. These data indicate that the combination of CD64-CR T cells and 376.96 mAb may have significant anti-tumor activity (FIG. 12C)

### Materials and Methods

The above Examples were performed using the following materials and methods.

### Antibodies and Reagents

Allophycocyanin-conjugated mouse anti-human CCR7 Clone 2L1A (566762), fluorescein isothiocyanate-conjugated mouse anti-human CD3 Clone UCHT1 (555332), PerCP-Cy™5.5-conjugated mouse anti-human CD4 Clone SK3 (332772), APC-conjugated mouse anti-human CD8 Clone RPA-T8 (555369), PerCP-Cy™5.5-conjugated mouse anti-human CD45RA Clone HI100 (563429), FITC-conjugated mouse anti-human CD62L Clone DREG-56 (555543), phycoerythrin (PE)-conjugated mouse anti-human CD64 Clone 10.1 (558592), FITC-conjugated mouse anti-human CD107A Clone H4A3 (555800), FITC-conjugated mouse anti-human IgG Clone G18-145 (555786), FITC-conjugated goat anti-mouse IgG (555988), FITC-conjugated anti-human CD279 (PD-1) clone MIH4 (557860), FITC-conjugated Annexin V (556420), propidium iodide (P1) staining solution (51-66211E), purified NA/LE mouse anti-human CD3 Clone UCHT1 (555329) and purified NA/LE mouse anti-Human CD28 Clone CD28.2 (555725) were purchased from BD Bioscience (San Jose, CA, USA). Anti-human B7-H3 (CD276) mAb 376.96 was developed and characterized as previously described. Anti-EGFR mAbs, cetuximab (Erbitux), and panitumumab (Vectibix) were obtained from Merck Serono (Darmstadt, Germany) and Amgen (Thousand Oaks, CA, USA), respectively. Human recombinant interleukin-7 (IL-7) (130-095-367) and interleukin-15 (IL-15) (130-095-760), human FcR blocking reagent (BR) (130-059-901), human CD14 microbeads (130-050-201), and human CD56 microbeads (130-050-401) were purchased from Miltenyi Biotec (Bergisch Gladbach, Germany). Ficoll-Paque PLUS density gradient media was obtained from GE Healthcare Life science (Marlborough, MA, USA). (3-(4,5-Dimethylthiazol-2-Y1)-2,5-diphenyltetrazolium bromide (MTT) was obtained from Sigma-Aldrich (Saint Louis, MO, USA). Retronectin (Recombinant Human Fibronectin) was purchased from Takara Bio (Saint-Germain-en-Laye, France).

### Cell Lines

The 293T (RRID: CVCL_0063) packaging cell line was cultured in Iscove's Modified Dulbecco's Medium (IMDM). The KRAS-mutated HCT-116 (RRID: CVCL_0291) and firefly luciferase-expressing HCT-116 (HCT-116-Luc) cells were maintained in Roswell Park Memorial Institute (RPMI)-1640. The human CRC HT-29 (RRID: CVCL_0320) and Caco-2 (RRID: CVCL_0025) cell lines were cultured in Dulbecco's Modified Eagle's Medium (DMEM) (Thermo Fisher Scientific, Waltham, MA, USA). All mentioned media were supplemented with 10% fetal bovine serum (FBS), 2 mM L-glutamine, 0.1 mg/ml streptomycin, and 100 U/ml penicillin to obtain cell culture complete media. BJ human skin fibroblasts (CVCL_3653) and human lung fibroblasts IMR-90 (CVCL_0347) were cultured in DMEM complete medium supplemented with 10% FBS (South American origin).). Human primary myoblasts were maintained in DMEM supplemented with primary skeletal muscle growth kit (ATCC® PCS-950-040TH). The 293T cells were provided by Dr. Gianpietro Dotti, University of North Carolina, Chapel Hill, NC, USA. The CRC HCT-116, HT-29, and Caco-2 cells were provided by Dr. Giulio Cesare Spagnoli, University of Basel, Switzerland. HCT-116-Luc cells were purchased from Caliper Life Sciences (Caliper Life Sciences, Inc., Hopkinton, MA, USA). The cell lines were passaged twice for a week and kept in culture for a maximum of 6-8 weeks.

### CD64-CR and B7-H3 Constructs

Three CD64 chimeric receptors (CRs) were designed. The molecules were synthesized by GeneArt Gene Synthesis service (Thermo Fisher Scientific, Waltham, MA, USA). Table 1 describes the leader sequences and the domains chosen for assembling the CD64-CRs. The amino acid sequence of the molcules is shown in FIG. 1F.

TABLE 1

| | Leader Sequence | Extracellular | Transmembrane | Intracellular |
|---|---|---|---|---|
| Construct name | (LS) | Domain (Ec) | Domain (Tm) | domain |
| CD64(Ec-Tm).28.ζ | Human CD64 | CD64 | CD64 | CD28 |
| | | | | CD3ζ |
| Hu-LS-CD64.28.ζ | Human CD64 | CD64 | CD28 | CD28 |
| | | | | CD3ζ |
| Ms-LS-CD64.28.ζ | Murine CD64 | CD64 | CD28 | CD28 |
| | | | | CD3ζ |

After synthesis, the gene cassettes of the chimer receptors were cloned into the NcoI and MluI sites of the SFG retroviral vector.

B7-H3.CAR, kindly provided by Gianpietro Dotti (UNC), was composed of scFv of the human B7-H3 mAb clone 376.96 fused to the transmembrane domain of CD8α and the intracellular motif of the CD28 and CD3ζ chain.

Amino acid sequences of selected CD64-CR constructs were synthesized for these studies as follows.

(MS-SP)-CD64.28.ζ
The amino acid sequence of (MS-SP)-CD64.28.ζ is:
(SEQ ID NO: 1)
MGILTSFGDDMWLLTTLLLWVPVGGQVDTTKAVITLQPPWVSVFQEETVTLHCEVLHLPGSSSTQW
FLNGTATQTSTPSYRITSASVNDSGEYRCQRGLSGRSDPIQLEIHRGWLLLQVSSRVFTEGEPLALRCHAWK
DKLVYNVLYYRNGKAFKFFHWNSNLTILKTNISHNGTYHCSGMGKHRYTSAGISVTVKELFPAPVLNASVTS
PLLEGNLVTLSCETKLLLQRPGLQLYFSFYMGSKTLRGRNTSSEYQILTARREDSGLYWCEAATEDGNVLKR
SPELELQVLGLQLPTPVWFHPGPSKPFWVLVVVGGVLACYSLLVTVAFIIFWVRSKRSRLLHSDYMNMTPRR
PGPTRKHYQPYAPPRDFAAYRSVDRVKFSRSADAPAYQQGQNQLYNELNLGRREEYDVLDKRRGRDPEM
GGKPRRKNPQEGLYNELQKDKMAEAYSEIGMKGERRRGKGHDGLYQGLSTATKDTYDALHMQALPPR
CD64.28.ζ,
The amino acid sequence of CD64.28.ζ is:
(SEQ ID NO: 8)
MGFLTTLLLWVPVDGQVDTTKAVITLQPPWVSVFQEETVTLHCEVLHLPGSSSTQWFLNGTATQTS
TPSYRITSASVNDSGEYRCQRGLSGRSDPIQLEIHRGWLLLQVSSRVFTEGEPLALRCHAWKDKLVYNVLYY
RNGKAFKFFHWNSNLTILKTNISHNGTYHCSGMGKHRYTSAGISVTVKELFPAPVLNASVTSPLLEGNLVTLS
CETKLLLQRPGLQLYFSFYMGSKTLRGRNTSSEYQILTARREDSGLYWCEAATEDGNVLKRSPELELQVLGL
QLPTPVWFHPGPSKPFWVLVVVGGVLACYSLLVTVAFIIFWVRSKRSRLLHSDYMNMTPRRPGPTRKHYQP
YAPPRDFAAYRSVDRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR*
CD64(EC-TM).28.ζ
The amino acid sequence of CD64(EC-TM).28.ζ is:
(SEQ ID NO: 15)
MGFLTTLLLWVPVDGQVDTTKAVITLQPPWVSVFQEETVTLHCEVLHLPGSSSTQWFLNGTATQTS
TPSYRITSASVNDSGEYRCQRGLSGRSDPIQLEIHRGWLLLQVSSRVFTEGEPLALRCHAWKDKLVYNVLYY
RNGKAFKFFHWNSNLTILKTNISHNGTYHCSGMGKHRYTSAGISVTVKELFPAPVLNASVTSPLLEGNLVTLS
CETKLLLQRPGLQLYFSFYMGSKTLRGRNTSSEYQILTARREDSGLYWCEAATEDGNVLKRSPELELQVLGL
QLPTPVWFHVLFYLAVGIMFLVNTVLWVTIVRSKRSRLLHSDYMNMTPRRPGPTRKHYQPYAPPRDFAAYR
SVDRVKFSRSAEPPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR*

In each of the above, the components are as follows:

(1) EXTRACELLULAR REGION OF THE HUMAN CD64
(SEQ ID NO: 3)
(QVDTTKAVITLQPPWVSVFQEETVTLHCEVLHLPGSSSTQWFLNGTATQ
TSTPSYRITSASVNDSGEYRCQRGLSGRSDPIQLEIHRGWLLLQVSSRVF
TEGEPLALRCHAWKDKLVYNVLYYRNGKAFKFFHWNSNLTILKTNISHNG
TYHCSGMGKHRYTSAGISVTVKELFPAPVLNASVTSPLLEGNLVTLSCET
KLLLQRPGLQLYFSFYMGSKTLRGRNTSSEYQILTARREDSGLYWCEAAT
EDGNVLKRSPELELQVLGLQLPTPVWFH);
(2) 6 amino acids from CD28 extracellular region
(SEQ ID NO: 4)
(PGPSKP);
(3) CD28 TRANSMEMBRANE
(SEQ ID NO: 5)
(FWVLVVVGGVLACYSLLVTVAFIIFWV);
(4) CD28 CYTOPLASMIC DOMAIN
(SEQ ID NO: 6)
(RSKRSRLLHSDYMNMTPRRPGPTRKHYQPYAPPRDFAAYRS);
(5) VD: restriction enzymes site;
(6) CD35 INTRACELLULAR MOTIF
(SEQ ID NO: 7)
(RVKFSRSAEPPAYQQGQNQLYNELNLGRREEYDVLDKRRGRDPEMGGKP
RRKNPQEGLYNELQKDKMAEAYSEIGMKGERRRGKGHDGLYQGLSTATKD
TYDALHMQALPPR);
(7) THE SP DERIVED FROM MOUSE CD64 (MS-SP)
(SEQ ID NO: 2)
(MGILTSFGDDMWLLTTLLLWVPVGG);
(8) SP DERIVED FROM HUMAN CD64 (HU-SP)
(SEQ ID NO: 9)
(MGFLTTLLLWVPVDG);
and
(9) CD64 TRANSMEMBRANE REGION
(SEQ ID NO: 18)
(VLFYLAVGIMFLVNTVLWVTI)

The sequence identifiers for the foregoing sequences are shown in Table 2.

TABLE 2
Sequence Listing

| SEQ ID | Construct/Component | Sequence |
|---|---|---|
| 1 | (MS-SP)-CD64.28.ζ | MGILTSFGDDMWLLTTLLLWVPVGGQVDTTKAVITLQPPWVSVFQE |
| | | ETVTLHCEVLHLPGSSSTQWFLNGTATQTSTPSYRITSASVNDSGE |
| | | YRCQRGLSGRSDPIQLEIHRGWLLLQVSSRVFTEGEPLALRCHAW |
| | | KDKLVYNVLYYRNGKAFKFFHWNSNLTILKTNISHNGTYHCSGMGK |
| | | HRYTSAGISVTVKELFPAPVLNASVTSPLLEGNLVTLSCETKLLLQR |
| | | PGLQLYFSFYMGSKTLRGRNTSSEYQILTARREDSGLYWCEAATE |
| | | DGNVLKRSPELELQVLGLQLPTPVWFHPGPSKPFWVLVVVGGVLA |
| | | CYSLLVTVAFIIFWVRSKRSRLLHSDYMNMTPRRPGPTRKHYQPYA |
| | | PPRDFAAYRSVDRVKFSRSADAPAYQQGQNQLYNELNLGRREEY |
| | | DVLDKRRGRDPEMGGKPRRKNPQEGLYNELQKDKMAEAYSEIGM |
| | | KGERRRGKGHDGLYQGLSTATKDTYDALHMQALPPR |
| 2 | SP DERIVED FROM | MGILTSFGDDMWLLTTLLLWVPVGG |
| | MOUSE CD64 (MS-SP) | |
| 3 | EXTRACELLULAR | QVDTTKAVITLQPPWVSVFQEETVTLHCEVLHLPGSSSTQWFLNGT |
| | REGION OF THE | ATQTSTPSYRITSASVNDSGEYRCQRGLSGRSDPIQLEIHRGWLLL |
| | HUMAN CD64 | QVSSRVFTEGEPLALRCHAWKDKLVYNVLYYRNGKAFKFFHWNSN |
| | | LTILKTNISHNGTYHCSGMGKHRYTSAGISVTVKELFPAPVLNASVT |
| | | SPLLEGNLVTLSCETKLLLQRPGLQLYFSFYMGSKTLRGRNTSSEY |
| | | QILTARREDSGLYWCEAATEDGNVLKRSPELELQVLGLQLPTPVWFH |
| 4 | 6 AMINO ACIDS | PGPSKP |
| | FROM CD28 | |
| | EXTRACELLULAR | |
| | REGION | |
| 5 | CD28 | FWVLVVVGGVLACYSLLVTVAFIIFWV |
| | TRANSMEMBRANE | |
| 6 | CD28 CYTOPLASMIC | RSKRSRLLHSDYMNMTPRRPGPTRKHYQPYAPPRDFAAYRS |
| | DOMAIN | |
| 7 | CD35 | RVKFSRSADAPAYQQGQNQLYNELNLGRREEYDVLDKRRGRDPE |
| | INTRACELLULAR | MGGKPRRKNPQEGLYNELQKDKMAEAYSEIGMKGERRRGKGHD |
| | MOTIF | GLYQGLSTATKDTYDALHMQALPPR |
| 8 | CD64.28.ζ, | MGFLTTLLLWVPVDGQVDTTKAVITLQPPWVSVFQEETVTLHCEVL |
| | | HLPGSSSTQWFLNGTATQTSTPSYRITSASVNDSGEYRCQRGLSG |
| | | RSDPIQLEIHRGWLLLQVSSRVFTEGEPLALRCHAWKDKLVYNVLY |
| | | YRNGKAFKFFHWNSNLTILKTNISHNGTYHCSGMGKHRYTSAGISV |
| | | TVKELFPAPVLNASVTSPLLEGNLVTLSCETKLLLQRPGLQLYFSFY |
| | | MGSKTLRGRNTSSEYQILTARREDSGLYWCEAATEDGNVLKRSPE |
| | | LELQVLGLQLPTPVWFHPGPSKPFWVLVVVGGVLACYSLLVTVAFII |
| | | FWVRSKRSRLLHSDYMNMTPRRPGPTRKHYQPYAPPRDFAAYRS |
| | | VDRVKFSRSADAPAYQQGQNQLYNELNLGRREEYDVLDKRRGRD |
| | | PEMGGKPRRKNPQEGLYNELQKDKMAEAYSEIGMKGERRRGKGH |
| | | DGLYQGLSTATKDTYDALHMQALPPR* |
| 9 | SP DERIVED FROM | MGFLTTLLLWVPVDG |
| | HUMAN CD64 (HU-SP) | |
| 10 | EXTRACELLULAR | QVDTTKAVITLQPPWVSVFQEETVTLHCEVLHLPGSSSTQWFLNGT |
| | REGION OF THE | ATQTSTPSYRITSASVNDSGEYRCQRGLSGRSDPIQLEIHRGWLLL |
| | HUMAN CD64 | QVSSRVFTEGEPLALRCHAWKDKLVYNVLYYRNGKAFKFFHWNSN |
| | | LTILKTNISHNGTYHCSGMGKHRYTSAGISVTVKELFPAPVLNASVT |
| | | SPLLEGNLVTLSCETKLLLQRPGLQLYFSFYMGSKTLRGRNTSSEY |
| | | QILTARREDSGLYWCEAATEDGNVLKRSPELELQVLGLQLPTPVWF |
| | | H |
| 11 | 6 AMINO ACIDS | PGPSKP |
| | FROM CD28 | |
| | EXTRACELLULAR | |
| | REGION | |
| 12 | CD28 | FWVLVVVGGVLACYSLLVTVAFIIFWV |
| | TRANSMEMBRANE | |
| 13 | CD28 CYTOPLASMIC | RSKRSRLLHSDYMNMTPRRPGPTRKHYQPYAPPRDFAAYRS |
| | DOMAIN | |
| 14 | CD3ζ | RVKFSRSADAPAYQQGQNQLYNELNLGRREEYDVLDKRRGRDPE |
| | INTRACELLULAR | MGGKPRRKNPQEGLYNELQKDKMAEAYSEIGMKGERRRGKGHD |
| | MOTIF | GLYQGLSTATKDTYDALHMQALPPR |
| 15 | CD64(EC-TM).28.ζ | MGFLTTLLLWVPVDGQVDTTKAVITLQPPWVSVFQEETVTLHCEVL |
| | | HLPGSSSTQWFLNGTATQTSTPSYRITSASVNDSGEYRCQRGLSG |
| | | RSDPIQLEIHRGWLLLQVSSRVFTEGEPLALRCHAWKDKLVYNVLY |
| | | YRNGKAFKFFHWNSNLTILKTNISHNGTYHCSGMGKHRYTSAGISV |
| | | TVKELFPAPVLNASVTSPLLEGNLVTLSCETKLLLQRPGLQLYFSFY |
| | | MGSKTLRGRNTSSEYQILTARREDSGLYWCEAATEDGNVLKRSPE |
| | | LELQVLGLQLPTPVWFHVLFYLAVGIMFLVNTVLWVTIVRSKRSRLL |
| | | HSDYMNMTPRRPGPTRKHYQPYAPPRDFAAYRSVDRVKFSRSAE |
| | | PPAYQQGQNQLYNELNLGRREEYDVLDKRRGRDPEMGGKPRRKN |
| | | PQEGLYNELQKDKMAEAYSEIGMKGERRRGKGHDGLYQGLSTAT |
| | | KDTYDALHMQALPPR* |
| 16 | SP DERIVED FROM | MGFLTTLLLWVPVDG |
| | HUMAN CD64 (HU-SP) | |
| 17 | EXTRACELLULAR | QVDTTKAVITLQPPWVSVFQEETVTLHCEVLHLPGSSSTQWFLNGT |
| | REGION OF THE | ATQTSTPSYRITSASVNDSGEYRCQRGLSGRSDPIQLEIHRGWLLL |
| | HUMAN CD64 | QVSSRVFTEGEPLALRCHAWKDKLVYNVLYYRNGKAFKFFHWNSN |
| | | LTILKTNISHNGTYHCSGMGKHRYTSAGISVTVKELFPAPVLNASVT |
| | | SPLLEGNLVTLSCETKLLLQRPGLQLYFSFYMGSKTLRGRNTSSEY |
| | | QILTARREDSGLYWCEAATEDGNVLKRSPELELQVLGLQLPTPVWF |
| | | H |
| 18 | CD64 | VLFYLAVGIMFLVNTVLWVTI |
| | TRANSMEMBRANE | |
| | REGION | |
| 19 | CD28 CYTOPLASMIC | RSKRSRLLHSDYMNMTPRRPGPTRKHYQPYAPPRDFAAYRS |
| | DOMAIN | |
| 20 | CD3ζ | RVKFSRSAEPPAYQQGQNQLYNELNLGRREEYDVLDKRRGRDPE |
| | INTRACELLULAR | MGGKPRRKNPQEGLYNELQKDKMAEAYSEIGMKGERRRGKGHD |
| | MOTIF | GLYQGLSTATKDTYDALHMQALPPR |
| 21 | FULL-LENGTH | MWFLTTLLLWVPVDGQVDTTKAVITLQPPWVSVFQEETVTLHCEVL |
| | HUMAN CD64 | HLPGSSSTQWFLNGTATQTSTPSYRITSASVNDSGEYRCQRGLSG |
| | (UNIPROT | RSDPIQLEIHRGWLLLQVSSRVFTEGEPLALRCHAWKDKLVYNVLY |
| | ACCESSION NO. | YRNGKAFKFFHWNSNLTILKTNISHNGTYHCSGMGKHRYTSAGISV |
| | P12314-1) | TVKELFPAPVLNASVTSPLLEGNLVTLSCETKLLLQRPGLQLYFSFY |
| | | MGSKTLRGRNTSSEYQILTARREDSGLYWCEAATEDGNVLKRSPE |
| | | LELQVLGLQLPTPVWFHVLFYLAVGIMFLVNTVLWVTIRKELKRKKK |
| | | WDLEISLDSGHEKKVISSLQEDRHLEEELKCQEQKEEQLQEGVHRK |
| | | EPQGAT |
| 22 | CD64 | VLFYLAVGIMFLVNTVLWVTIV |
| | TRANSMEMBRANE | |
| | DOMAIN WITH A | |
| | VALINE | |
| 23 | CD28 AND CD3-ZETA | RSKRSRLLHSDYMNMTPRRPGPTRKHYQPYAPPRDFAAYRSVDR |
| | SIGNALING DOMAIN | VKFSRSAEPPAYQQGQNQLYNELNLGRREEYDVLDKRRGRDPEM |
| | | GGKPRRKNPQEGLYNELQKDKMAEAYSEIGMKGERRRGKGHDGL |
| | | YQGLSTATKDTYDALHMQALPPR |

### Retrovirus Production

Retroviral supernatant production was performed as previously described^{28,29}.

Briefly, the 293T packaging cells were cotransfected with CD64-CR construct plus the Peg-Pam and the RDF vectors encoding MoMLV gag-pol proteins and RD114 envelope, respectively. Cell supernatant containing retroviral particles were harvested 48 h and 72 h after transfection. RetroNectin-coated non-tissue culture treated 24-well plates were preloaded with 1 ml/well of viral supernatant and used for T cell transduction.

### PBMCs Isolation and Human T Cell Transduction

Blood samples were obtained from the Department of Transfusion Medicine of the Tor Vergata Hospital, Rome. Peripheral blood mononuclear cells (PBMCs) were isolated from buffy coats with Ficoll-paque Plus reagent according to the manufacturer's instruction and CD56⁺ NK cells were removed utilizing the human CD56 microbeads. For activation, T cells were cultured for 72 hours in a non-tissue culture treated 24-well plate precoated with 1 μg/ml of anti-human CD3 and 1 μg/ml of anti-human CD28 mAb mix. Then, proliferating T cells (0.5×10⁶/well) were seeded into a retroviral-preloaded 24-well plate for 72 h at 37° C. in 5% CO₂. After transduction, the T cells were expanded in RPMI-1640 complete medium supplemented with 10 ng/ml of IL-7 and 5 ng/ml of IL-15 for further analysis. All the experiments were conducted without the addition of the cytokine to the medium.

### Magnetic Sorting of CD14+ Cells

CD14+ monocytes were selected from PBMCs using human CD14 microbeads (Miltenyi) according to the manufacturers instruction. Briefly, a pellet of 30×10⁶ PBMCs was incubated with 60 μl of anti-human CD14 microbeads for 15 min at 4° C. Then, the stained cells were flowed through a BS column (Miltenyi) for a positive selection. Then CD14+ adherent cells were eluted by mechanical pressure. The average purity of the recovered CD14+ cells was higher than 95%.

### Binding Assay of the Monoclonal Antibody Fc on the CD64-CR

CD64-CR transduced T cells were pre-incubated for 15 min at 4° C. with or without 54 of FcR blocking reagent (FcR BR) and then incubated with 10 μg/ml of the anti-human EGFR cetuximab (IgG1), panitumumab (IgG2) or mouse anti-human B7-H3 mAb (376.96) (IgG2A) for 30 min at 4° C. Then, cells were washed and stained with FITC-conjugated mouse anti-human IgG or FITC-conjugated goat anti-mouse IgG for 30 min at 4° C. In dose-response experiments, the cells were incubated for 30 min at 4° C. with increasing amounts of cetuximab, panitumumab, and 376.96 mAb and then stained with the above-mentioned secondary antibodies. In the competition test, the CD64-CR T cells were incubated with different doses of heat-inactivated human plasma for 30 min at 4° C. or 37° C. and then 10 μg/ml of 376.96 mAb was added for 30 min at 4° C. or 37° C. The analysis of the mAb binding on the CD64-CR T cell surface was performed by flow cytometry.

### Flow Cytometry

The assessment of CD64-CR expression on T cells and phenotypic analysis of transduced T cell subsets was performed by flow cytometry. After transduction, T cells were incubated for 30 min at 4° C. with mAbs specific for CD3, CD64, CD45RA, CD62L, CCR7 conjugated with FITC, PE, PerCP-Cy5.5, FITC, and APC fluorochromes, respectively. After staining, cells were washed and fixed with 1% of the formaldehyde-PBS solution. Samples were acquired with a 2-laser BD FACSCalibur™ flow cytometer using BD CellQuest software (Becton Dickinson, Franklin Lakes, NJ, USA). All data were analyzed using Tree Star, Inc. FlowJo software.

### In Vitro Coculture Experiments

MTT assay. CRC or non-tumor target cells (7×10³) were cocultured into flat-bottom 96-well plates with CD64-CR T cells at different effector: target (E:T) ratios in the presence or absence of 1 μg/ml of cetuximab or anti-human B7-H3 (376.96) mAb, for 72 hours at 37° C. An experimental triplicate was performed for each condition. After incubation, target cell viability was determined by MTT assay as previously detailed (Arriga et al., Int J Cancer 2020; 146:2531-8; Caratelli et al., Int J Cancer 2020; 146:236-47).

Bioluminescent Imaging (BLI). HCT-116-luc cells were seeded in 96-well microplates in triplicate (n=3) and incubated with transduced or non-transduced T cells at various E:T ratios. After 3-day coculture, the cell culture medium was supplemented with D-luciferin (PerkinElmer) dissolved in PBS (150 μg/mL) for 10 min and analysis was performed using the IVIS® Lumina II platform (PerkinElmer, Waltham, MA, USA). Photons emitted from luciferase-expressing cells in selected regions of interest (ROI) were quantified using the Living Image® software.

Target Cell depletion. HCT-116, HT-29, and CaCo-2 CRC cell lines (0.02×10⁶/well) were cocultured in triplicate in 96-well plate with CD64-CR transduced or non-transduced control T cells at various E:T ratio for approximately 18 hours. Then, non-adherent effector T cells were removed and the remaining adherent CRC target cells were enumerated by trypan blue-exclusion cell count.

Flow-cytometry cytotoxicity assay. HCT-116 target cells (0.2×10⁶) were seeded in a 24-well plate. After 6-hour incubation, 0.4×10⁶ CD64-CR transduced or non-transduced T cells (E:T=2:1) were added to the wells with or without 1 μg/ml of cetuximab or 376.96 mAb for approximately 18 hours at 37° C. To evaluate the target cell death levels, the cell coculture were collected and stained with APC-conjugated mouse anti-human CD3, FITC-conjugated annexin V and PI solution for 15 min at room temperature, in the dark. The percentage of target cell apoptosis/necrosis was determined by flow cytometry.

### CD107A Release Test

CD64-CR T cells (0.4×10⁶) were incubated with HCT116 (0.2×10⁶) cells in 96-well microplates in the presence or absence of 1 μg/ml of cetuximab, panitumumab, 376.96. To maximize CD107A detection, FITC-conjugated mouse anti-human CD107A mAb was included during coculture with the addition of 2 μM of the secretion inhibitor monensin used for blocking CD107A antibody acidification and degradation. After a 4-hour-incubation at 37° C., cells were stained with PE-conjugated mouse anti-human CD64 and analyzed by flow cytometry.

### Immunofluorescence and Confocal Microscopy

HCT116 colon cancer cells were cocultured with CD64-CR expressing T cells for 30 min, fixed for 10 minutes with 3% (w/v) formaldehyde in PBS, permeabilized 10 minutes with 0.15% Triton, blocked for 1 hr 5% BSA, and immunostained with anti-EGFR (1:20, cell signaling 08/2019 D3861), anti-CD64 (1:40, BD 555525), overnight at 4° C. in 1% BSA. Cells were incubated with the following secondary antibodies (diluted in 1% BSA) at room temperature for 1 h: Cy3-conjugated donkey anti-mouse and Cy5-conjugated donkey anti-rabbit antibodies (Jackson ImmunoResearch Laboratories). Then, cells were immunostained 2 h at 37° C. with FITC conjugated anti-human perforin antibody (1:10). The DNA was counterstained with 0.4 mg/ml DAPI (33258; Sigma). Slides were mounted in 50% glycerol and analyzed within 24 h. Images were recorded by using a Zeiss LSM 880 confocal laser scanning microscope equipped with a 60X/1.23 NA oil immersion objective, and laser 405, 488, 543, 633 nm were used to excite the fluorophores.

### Statistical Analysis

Data sets were analyzed using Graphpad Prism Software. Unpaired two-tailed T-test and two-way ANOVA tests were used. Differences with a p-value <0.05 were considered to be statistically significant.

### Uses

The human Fc receptor CD64, as described herein, has been linked to the stimulatory molecules CD28 and CD3. This construct is transduced to human T cells. The CD64 T cells in combination with the B7-H3-specific mAb 376.96 acquire the ability to specifically eliminate human tumor cells expressing B7-H3. Therefore CD64 T cells in combination with mAb 376.96 are useful for immunotherapy of tumors which express B7-H3. This combination can be extended to other tumor antigen-specific mAbs. Table 3 shows an exemplary list cancer cell lines tested positive for B7-H3 antigen.

TABLE 3
EXEMPLARY LIST OF B7-H3
ANTIGEN EXPRESSING CELLS

| | |
|---|---|
| U937: | AML |
| ML-2: | AML |
| KMN-1: | MULTIPLE MYELOMA |
| HCT-116: | COLORECTAL CARCINOMA |
| HT-29: | COLORECTAL CARCINOMA |
| MDA-MB-231: | BREAST CARCINOMA |
| MDA-MB-468: | BREAST CARCINOMA |
| A549: | NON SMALL CELL LUNG CARCINOMA |
| U87: | GLIOBLASTOMA MULTIFORME (GB) |
| U373: | GB |
| CSC2: | GB (CANCER STEM CELLS) |
| CSC3: | GB (CANCER STEM CELLS) |
| FADU: | HEAD AND NECK CANCER |
| A-253: | HEAD AND NECK CANCER |

Nucleic acids encoding any of the CRs described herein are useful in the invention, including the FcgRI (CD64) chimeric receptor or a binding domain thereof (such as the FcγRI D2 domain C-strand, C’-strand, and/or the C′E loop) resulting from the fusion of the extracellular or extracellular plus transmembrane domain of CD64 with a T cell costimulatory molecule joint to the T cell receptor chain. This molecule is cloned into an expression vector that is transduced or transfected in a host cell in combination with the 396.96 monoclonal antibody (mAb) with specificity for the B7-H3 antigen for immunotherapeutic applications including cancer and other diseases.

The invention has many applications in the field of cancer therapy. Products described herein are useful for the implementation of adoptive T cell transfer-based immunotherapies for the treatment of a wide range of hematologic and solid B7-H3 positive tumors (such as colorectal cancer, breast cancer, non-small cell lung cancer, head and neck cancer, glioblastoma, leukemia, myeloma).

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations described herein following, in general, the principles described herein and including such departures from the invention that come within known or customary practice within the art to which the invention pertains and may be applied to the essential features hereinbefore set forth, and follows in the scope of the claims.

Other embodiments are within the claims.

What is claimed is:

1. A chimeric receptor comprising
a.) an extracellular ligand binding domain of CD64;
b.) a transmembrane domain; and
c.) an intracellular domain.

2. The chimeric receptor of claim 1, further comprising a signal peptide.

3. The chimeric receptor of claim 2, wherein the signal peptide is a mouse CD64 signal peptide.

4. The chimeric receptor of claim 3, wherein the mouse CD64 signal peptide is humanized.

5. The chimeric receptor of claim 1, wherein the extracellular ligand binding domain comprises D1, D2, and D3 of CD64.

6. The chimeric receptor of claim 1, wherein the transmembrane domain comprises a CD64 transmembrane domain.

7. The chimeric receptor of claim 1 wherein the domain further comprises a hinge region.

8. The chimeric receptor of claim 1, wherein the transmembrane domain comprises a CD28 transmembrane domain.

9. The chimeric receptor of claim 1, wherein the intracellular domain comprises a CD28 intracellular domain.

10. The chimeric receptor of claim 1, wherein the intracellular domain comprises a CD28-CD3ζ intracellular domain.

11. The chimeric receptor of claim 1, wherein the chimeric receptor comprises CD3ζ.

12. The chimeric receptor of claim 1, wherein the chimeric receptor binds to a molecule comprising Fc.

13. The chimeric receptor of claim 12, wherein the molecule comprising Fc is an IgG antibody.

14. The chimeric receptor of claim 13, wherein the IgG antibody is IgG1 or IgG3.

15. A nucleic acid comprising a nucleotide sequence encoding a chimeric receptor of any one of claims 1-14.

16. The nucleic acid of claim 15, wherein the nucleic acid is an RNA or a cDNA molecule.

17. A vector comprising the nucleic acid of claim 15 or claim 16.

18. The vector of claim 17, wherein the vector is an expression vector.

19. The vector of claim 18, wherein the vector is a viral vector.

20. The vector of claim 19, wherein the viral vector is a retroviral vector.

21. A cell, comprising a nucleic acid of claim 15 or claim 16, or a vector of any one of claims 17-20.

22. The cell of claim 21, wherein the cell is an immune cell.

23. The cell of claim 22, wherein the immune cell is a T cell.

24. The cell of any one of claims 21-23, wherein the cell is a T cell.

25. A pharmaceutical composition, comprising (a) a nucleic acid of claim 15 or claim 16, a vector of any one of claims 17-20, or a cell of any one of claims 21-24, and (b) a pharmaceutically acceptable carrier.

26. The pharmaceutical composition of claim 25, wherein the composition further comprises an Fc-containing therapeutic agent.

27. The pharmaceutical composition of claim 26, wherein the Fc-containing therapeutic agent is an Fc fusion protein or an antibody.

28. The pharmaceutical composition of claim 27, wherein the antibody binds to a cell surface antigen.

29. The pharmaceutical composition of claim 27 or 28, wherein the antibody binds epidermal growth factor receptor.

30. The pharmaceutical composition of claim 29, wherein the antibody is Cetuximab.

31. The pharmaceutical composition of claim 27 or 28, wherein the antibody binds B7-H3.

32. The pharmaceutical composition of claim 31, wherein the antibody is anti-B7-H3 376.96 mAb.

33. The pharmaceutical composition of claim 31, wherein the antibody is Enoblituzumab.

34. A kit, comprising:
a first pharmaceutical composition that comprises (i) a nucleic acid of claim 15 or claim 16, a vector of any one of claims 17-20, or a cell of any one of claims 21-24, and (ii) a pharmaceutically acceptable carrier.

35. The kit of claim 34, further comprising a second pharmaceutical composition that comprises an Fc-containing therapeutic agent and a pharmaceutically acceptable carrier.

36. The kit of claim 35, wherein the Fc-containing therapeutic agent is an Fc fusion protein or an antibody.

37. The kit of claim 36, wherein the antibody binds to a cell surface antigen.

38. The kit of claim 36 or 37, wherein the antibody binds epidermal growth factor receptor.

39. The kit of claim 38, wherein the antibody is Cetuximab.

40. The kit of claim 36 or 37, wherein the antibody binds B7-H3.

41. The kit of claim 40, wherein the antibody is anti-B7-H3 376.96 mAb.

42. The kit of claim 40, wherein the antibody is Enoblituzumab.

43. A pharmaceutical composition for use in killing tumor cells or for treating cancer in a subject, the pharmaceutical composition comprising an effective amount of immune cells that express a chimeric receptor of any one of claims 1-14 and a pharmaceutically acceptable carrier.

44. A pharmaceutical composition for use in enhancing antibody-dependent cell-mediated cytotoxicity (ADCC) or antibody-dependent cellular phagocytosis (ADCP) or for enhancing efficacy of an antibody-based immunotherapy in a subject, the pharmaceutical composition comprising an effective amount of immune cells that express a chimeric receptor of any one of claims 1-14 and a pharmaceutically acceptable carrier.

45. The pharmaceutical composition for use of claim 43 or 44, wherein the immune cells are natural killer cells, macrophages, neutrophils, eosinophils, T cells, or a combination thereof.

46. The pharmaceutical composition for use of claim 45, wherein the immune cell is a T cell.

47. The pharmaceutical composition for use of claim 46, wherein the host immune cells are autologous.

48. The pharmaceutical composition for use of claim 46, wherein the host immune cells are allogeneic.

49. The pharmaceutical composition for use of any one of claims 43-48, wherein the immune cells are activated, expanded, or both ex vivo.

50. The pharmaceutical composition for use of any one of claims 43-49, wherein the subject has been treated or is being treating with an Fc-containing therapeutic agent.

51. The pharmaceutical composition for use of claim 50, wherein the Fc-containing therapeutic agent is a therapeutic antibody or a Fc fusion protein.

52. The pharmaceutical composition for use of claim 51, wherein the Fc-containing therapeutic agent is an antibody.

53. The pharmaceutical composition for use of claim 52, wherein the antibody binds to a cell surface antigen.

54. The pharmaceutical composition for use of claim 52 or 53, wherein the antibody binds epidermal growth factor receptor.

55. The pharmaceutical composition for use of claim 54, wherein the antibody is Cetuximab.

56. The pharmaceutical composition for use of claim 52 or 53, wherein the antibody binds B7-H3.

57. The pharmaceutical composition for use of claim 56, wherein the antibody is anti-B7-H3 376.96 mAb.

58. The pharmaceutical composition for use of claim 56, wherein the antibody is Enoblituzumab.

59. The pharmaceutical composition for use of any one of claims 50-58, wherein the subject is a human patient suffering from a cancer and the Fc-containing therapeutic agent is for treating the cancer.

60. The pharmaceutical composition for use of claim 59, wherein the cancer includes cells expressing epidermal growth factor.

61. The pharmaceutical composition for use of claim 59, wherein the cancer includes cells expressing B7-H3.

62. The pharmaceutical composition for use of claim 59, wherein the cancer is a solid cancer or a hematopoietic cancer.

63. The pharmaceutical composition for use of claim 59, wherein the cancer is a carcinoma, lymphoma, a sarcoma, blastoma, a leukemia, or a cancer stem cell.

64. The pharmaceutical composition for use of claims 60-63, wherein the cancer is acute myeloid leukemia, breast cancer, lung cancer, non small cell lung carcinoma, gastrointestinal cancer, colorectal cancer, head and neck cancer, and glioblastoma multiforme, and glioblastoma multiforme cancer stem cells.

65. The pharmaceutical composition for use of any one of claims 43 and 59-64, wherein the subject's cancer has been irradiated or is being irradiated.

66. The pharmaceutical composition for use of any one of claims 43-65, wherein the subject has one or more tumors and the immune cells migrate to the one or more tumors.

67. A method for preparing immune cells expressing a chimeric receptor, comprising:
(i) providing a population of immune cells;
(ii) introducing into the immune cells a nucleic acid encoding a chimeric receptor of any one of claims 1-14; and
(iii) culturing the immune cells under conditions allowing for expression of the chimeric receptor.

68. The method of claim 67, wherein the population of immune cells are derived from peripheral blood mononuclear cells (PBMC).

69. The method of claim 67 or claim 68, wherein the immune cells are natural killer cells, macrophages, neutrophils, eosinophils, T cells, or a combination thereof.

70. The method of any one of claims 67-69, wherein the immune cells are derived from a human patient.

71. The method of claim 70, wherein the human patient is a cancer patient.

72. The method of any one of claims 67-71, wherein the nucleic acid is a viral vector.

73. The method of claim 72, wherein the viral vector is a retroviral vector.

74. The method of any one of claims 67-71, wherein the nucleic acid is an RNA molecule.

75. The method of any of claims 67-74, wherein the vector is introduced into the immune cells by retroviral transduction or electroporation.

76. A method of treating cancer in a subject, comprising administering to the subject an effective amount of the nucleic acid molecule of any of claims 15-16, the vector of any of claims 17-20, the cell of any of claims 21-24 and/or a pharmaceutical composition of any of claims 25-33, thereby treating cancer in the subject.

77. A method of enhancing ADCC or ADCP or for enhancing efficacy of an antibody-based immunotherapy in a subject having a cancer, comprising administering to the subject an effective amount of the nucleic acid molecule of any of claims 15-16, the vector of any of claims 17-20, the cell of any of claims 21-24 and/or a pharmaceutical composition of any of claims 25-33, thereby enhancing ADCC or ADCP or enhancing efficacy of the antibody-based immunotherapy in the subject.

78. The method of claim 76 or 77, wherein the subject has been treated or is being treating with an Fc-containing therapeutic agent.

79. The method of claim 78, wherein the Fc-containing therapeutic agent is a therapeutic antibody or a Fc fusion protein.

80. The method of claim 79, wherein the Fc-containing therapeutic agent is an antibody.

81. The method of claim 80, wherein the antibody binds to a cell surface antigen.

82. The method of claim 80 or 81, wherein the antibody binds epidermal growth factor receptor.

83. The method of claim 82, wherein the antibody is Cetuximab.

84. The method of claim 80 or 81, wherein the antibody binds B7-H3.

85. The method of claim 84, wherein the antibody is anti-B7-H3 376.96 mAb.

86. The method of claim 84, wherein the antibody is Enoblituzumab.

87. The method of any one of claims 78-86, wherein the Fc-containing therapeutic agent is for treating the cancer.

88. The method any one of claims 76-87, wherein the cancer includes cells expressing epidermal growth factor.

89. The method any one of claims 76-88, wherein the cancer includes cells expressing B7-H3.

90. The method any one of claims 76-89, wherein the cancer is a solid cancer or a hematopoietic cancer.

91. The method any one of claims 76-90, wherein the cancer is a carcinoma, lymphoma, a sarcoma, blastoma, a leukemia, or a cancer stem cell.

92. The method any one of claims 76-91, wherein the subject's cancer has been or is being irradiated.

93. The method any one of claims 76-92, wherein the subject has one or more tumors and the immune cells migrate to the one or more tumors.

94. A chimeric receptor comprising
a.) an extracellular ligand binding domain of CD64, wherein the extracellular ligand binding domain of CD64 comprises an amino acid sequence having at least 80%, at least 85%, at least 90%, at least 95%, at least 96%, at least 97%, at least 98%, or at least 99% sequence identity to the amino acid sequence of SEQ ID NO:3;
b.) a transmembrane domain; and
c.) an intracellular domain.

95. A chimeric receptor comprising
a.) an extracellular ligand binding domain of CD64, wherein the extracellular ligand binding domain of CD64 comprises one or more of the following: (i) a CD64 D2 domain C-strand (Tyr-133-Leu-136); (ii) a CD64 C′-strand (Lys-142-His-148); and/or (iii) a CD64 C′E loop (His-148-Trp-149);
b.) a transmembrane domain; and
c.) an intracellular domain.

96. The chimeric receptor of claim 95, wherein the CD64 D2 domain C-strand comprises amino acid residues Tyr-133 to Leu-136 of CD64 (YNVL).

97. The chimeric receptor of claim 95 or 96, wherein the CD64 C′-strand comprises amino acid residues Lys-142 to His-148 of CD64 (KAFKFFH).

98. The chimeric receptor of any one of claims 95-97, wherein the CD64 C′E loop comprises amino acid residues His-148 and Trp-149 of CD64 (HW).

99. The chimeric receptor of any one of claims 95-98, wherein the extracellular ligand binding domain of CD64 comprises (i) and (ii), (ii) and (iii), (i) and (iii), or (i), (ii), and (iii).

100. The chimeric receptor of any one of claims 96-99, wherein the numbering of the amino acid residues is relative to SEQ ID NO:21.

101. The chimeric receptor of any one of claims 95-100, wherein the extracellular ligand binding domain of CD64 comprises an amino acid sequence having at least 80%, at least 85%, at least 90%, at least 95%, at least 96%, at least 97%, at least 98%, or at least 99% sequence identity to the amino acid sequence of SEQ ID NO:3.
